SEQUENCE LISTING (33 sequences)

SEQ ID NO: 1 - DNA, 2786 bp, Homo sapiens
cgtggtggcgcacgcgtgtagtcccagctacttgggaggctgaggcagaagaatcccttgaacccaggaggcggaggttgcagtaagccaagattgtcactgcactccagcctgggcgaaagaggaagactccatctcaaaaaaaagaaaaaagaaatttcatggttatgcaactcttatttatgatcagaaaaatggacattttgtgatttaactctgtaacatgtttcatgtagtaaaaatataataaaactattaatcatctagcttgggagagataggagaaagacattactgtcactagtcaaattatatatcttttactatccaccaaaaatctcttctgatttctggttagaaggcatactattaattgataagaaaataaaactgaaggcctctaacatatcacagggtaataagaatatagggaaagttagttcaatagtttaaattaaagcacacttcttacagtatagaactagtcgggcttttatgccttgttttagttcttactcttcctttaactctttttctgttgatgtaatttacattaatgcttaagagtgaactttttaagtgtgggtaaaaacgaaataattacttacaaagtttaattcttccatttcctttgagagaggaaagttatggaaaagcagctcttatctaaagcaaagagcccacagattgatttcattggccctggatgtatttaatggatttttactatgcacataatttccagaagcattgttatttatttattaattataaatttagtgtaaccatttcatagggttacacagaactacccagttgtgcatgtctgatgtaatttcacatatgaatgtatgaattacttgtcttattcatgttgatacagcctcagtccatggcgcatccgtgtggggggaccccaacatacccagaatcacagatatttttcccaactattcatgaacgtccagtttctttttcaccacctcccacctgcccaccgaaagtagccatttcccagcggcgtaagagcacctccttcctggaagcccaaactcaccacttccaacccctgctgaggactgttggccaaagtcttcttccacctggtggcagcccaactaactggacaccagaggccgtagttatgttgggtactacagccagtagagtaactggagagtcatgtgagatacaggtccatcctatgtttgaaccatctcaagtttacagtgactatagacctggactagtacttccagaagaagctcactattttattcctcaggaagcagtgtatgtagctggggtacattaccaggcccgggtggcagaacagtatgagggcattccatacaactcatcagtactgtcaagtcctatgaaacagatacctgaacagaagccagtacaagggggccctacttcaagttctgtctttgaatttccatctggacaggctttcctggtaggacaccttcagaatctaagattagattctggattgggtccgggatctcccctctctagtatttctgcacctatcagtacagatgctacacgtttgaaatttcaccctgtctttgttcctcattctgcgcctgctgtgttaactcataacaatgagagcagaagcaactgtgtatttgaatttcatgttcacacaccaagctcctcttcaggagaaggaggtggaattttacctcagcgtgtttaccgaaatcggcaggttgcagtggacttgaatcaagaagaactgcctcctcaatcagttggattacatggctacttgcagcctgtgactgaagaaaagcataattaccatgccccagaattgaccgtttctgtggtagagcctatcggacagaactggccaataggaagcccagaatattccagtgattcctcacaaatcacttcttcagaccccagtgattttcagtcacctccccctacagggggagcagctgcaccttttggctctgacgtctcaatgccctttatccatctgcctcagacagtgttacaagaatccccacttttcttctgtttcccccaaggaaccacatctcagcaggtcttaactgcctcattttcttcaggaggatctgcacttcatccacaggttataggaaaacttccacaattattttaaactaccctactttgcaccataacatttaaattttctattccttatttccctgaatcatggattttggagaaatattgtttaattttatcagtagagtttccccatctttggggggttgtgaactacatatatgcatttaaaaacaaaatgtgagagaagctacctgatttacctattatatgtgaaaaccagtggaaaaaacacaaaaactagaattttagtcattcttcacaatcacgacttctatgcacgttattttcaaccagtagtgaaaatgcaagtgtatgtaatgtatggttgacccagcattatttaggaatacaaatcttaagtattactttcttcctccaaacaagtttttaaaaaataggataaattttttttctataaaatataaaacatggaaaatagggaatgctgtttttgaggtaatattaataatacacagaattttcattagtgtcgaaggatctaaaaagacaaagtatatcatgggaataaaaaaagatagaaaaggaaacagtttaggaatttgccttaacaaatgaaaatgcctttttaaaatggcatcagtcaagcaagttgctgtgcattattatatgtccaaataaaatgctaattcataaaattaagg

SEQ ID NO: 2 - protein, 434 aa, Homo sapiens
MYELLVLFMLIQPQSMAHPCGGTPTYPESQIFFPTIHERPVSFSPPPTCPPKVAISQRRKSTSFLEAQTHHFQPLLRTVGQSLLPPGGSPTNWTPEAVVMLGTTASRVTGESCEIQVHPMFEPSQVYSDYRPGLVLPEEAHYFIPQEAVYVAGVHYQARVAEQYEGIPYNSSVLSSPMKQIPEQKPVQGGPTSSSVFEFPSGQAFLVGHLQNLRLDSGLGPGSPLSSISAPISTDATRLKFHPVFVPHSAPAVLTHNNESRSNCVFEFHVHTPSSSSGEGGGILPQRVYRNRQVAVDLNQEELPPQSVGLHGYLQPVTEEKHNYHAPELTVSVVEPIGQNWPIGSPEYSSDSSQITSSDPSDFQSPPPTGGAAAPFGSDVSMPFIHLPQTVLQESPLFFCFPQGTTSQQVLTASFSSGGSALHPQVIGKLPQLF

SEQ ID NO: 3 - protein, 206 aa, Homo sapiens
MYELLVLFMLIQPQSMAHPCGGTPTYPESQIFFPTIHERPVSFSPPPTCPPKVAISQRRKSTSFLEAQTHHFQPLLRTVGQSLLPPGGSPTNWTPEAVVMLGTTASRVTGESCEIQVHPMFEPSQVYSDYRPGLVLPEEAHYFIPQEAVYVAGVHYQARVAEQYEGIPYNSSVLSSPMKQIPEQKPVQGGPTSSSVFDFHLDRLSW

SEQ ID NO: 4 - protein, 318 aa, Homo sapiens
MYELLVLFMLIQPQSMAHPCGGTPTYPESQIFFPTIHERPVSFSPPPTCPPKVAISQRRKSTSFLEAQTHHFQPLLRTVGQSLLPPGGSPTNWTPEAVVMLGTTASRVTGESCEIQVHPMFEPSQVYSDYRPGLVLPEEAHYFIPQEAVYVAGVHYQARVAEQYEGIPYNSSVLSSPMKQIPEQKPVQGGPTSSSVFEFPSGQAFLVGHLQNLRLDSGLGPGSPLSSISAPISTDATRLKFHPVFVPHSAPAVLTHNNESRSNCVFEFHVHTPSSSSGEGGGILPQRVYRNRQVAVDLNQEELPPQISWITWLLAACD

SEQ ID NO: 5 - protein, 314 aa, Homo sapiens
MYELLVLFMLIQPQSMAHPCGGTPTYPESQIFFPTIHERPVSFSPPPTCPPKVAISQRRKSTSFLEAQTHHFQPLLRTVGQSLLPPGGSPTNWTPEAVVMLGTTASRVTGESCEIQVHPMFEPSQVYSDYRPGLVLPEEAHYFIPQEAVYVAGVHYQARVAEQYEGIPYNSSVLSSPMKQIPEQKPVQGGPTSSSVFEFPSGQAFLVGHLQNLRLDSGLGPGSPLSSISAPISTDATRLKFHPVFVPHSAPAVLTHNNESRSNCVFEFHVHTPSSSSGEGGGILPQRVYRNRQVAVDLNQEELPPQSVGLHGYL

SEQ ID NO: 6 - DNA, 5562 bp, Homo sapiens
gtaagtaaatgcttaagagcatgtaatactacaatgagagccaatggatagtctgctaaattaaaattctttgtacaaaccaagtaacagtatgagtctgaagtagaaaatataaaatgctgtgtctctaaagccttattatagagtaggatatagacattaatgtgtgggcattagctgaactgcctacatgaagcagctatgtgcaagatttagattagtctgcatttttactaaagggaacgctctctttagcacttaagatgttgaaactattcacagcaacaaaagctggacatacaaatgttttgcgggtggggcgagaagagtttttcaaaagcttgttaaaatgattactaagtatttcctgtaagattccctattgtacaaaacacaggtaagaccttcaaaccattatagaatgctgcatgtgagtactctagattgagggaatcctttatttgaatgactgtttaatttagcgcttgtgttgagttaatcatcagttgctgtgtgtgttcctgccttttaaagtctgtgttggacacaagttccttccccagtatatgttcttttgctttttccctttagttggtaactgttttattactgatttattttcagaatcaacttatagaaattatttcctgaaacattttatgcttgtatacacatacacacacacaattatcctgtgttttacattagacactcttgtttctcatatagagaatgttaaagtagatcatcagataataccccatttcttttgttgacctcagaggaaaataaaatacgtcatctgcctctgaaaggtattagaaaataagtaacttgtcactgtacagcacttgtttagtgagatctttaagcagacagattttccccttctcagaaagcttttaatctaaactttgtggaattcttgctcttctttaaatgaaaatctttctctgttaagaaaatacttctgcttgttcgattttttatcctacagcttggaataaaagatgttcatttgtttcagagtccgtttaatcctgctttggctaggtctcaaaatgaatggccacagtaacacaaataaaaaactaatatactaactttttaaaacaatctactttctgtctttaaaataattgagggtagaatagaagaactttataaagactgcacagaaaatatatttctgtgttctgacaaatattacttgtcagcatttcttgtcagtttctacctttaattgttttttttctggattattataactagattttgctaagactttttttttaaaattttctcttgtctatactcatcatgggggagcattaggtctctattttagaaagcaagtagaatgacatgaagatattatttgagccattaatctagctatatttattgatgctctaaaagcagacgttcttgctttttaatgacagtattctatggaggtattatgaagaattattaaatattgagaatttaaaaatttgtctaattctattcttgtctcatttcaggaatgtggcttacaataatatggacaaattataagtgaaattttagaaattagaataaaagaaataaggctagagagaaatcaaaacaggaatgaaactaaacaatcatatgatgatatgtaaatacttggtattggtgggccacagatttggcccaaagcattctacacagccaagttttggaaatgaaaacatagtttcatagttcacaacatctaaaaggaaaataacagtcttattcaagacaacctttatggtattgatcatactgccttatagccatattaaaccctgtgcctgaacatttttttaagtttttttttttttttttgacagcgtctcactctgtctcccaggtgggaatacagtggcgtgatcttggctcactgcaactttcacctcctgggttcaagtgattctcctgcctcagcctccccagtagctaagattacaggcacccatcaccacgcccagctaatttttgtattttttttagtagagacacagtttcaccgtgttggctaggctggtcttgaactcttgacctcaagtgatccacctgcctcggcctcccaaagtgccaggattacaggtgtgagccaccatgcccggcctaaattattatttagagattcagattgaagatattctcccattgattactttgtaaaagtacaaaatatttcctatatccagttgtgaatttcttctagaaagctaaactttttcctagtagatcatgacttttttacctttacaaataaagagtggcaagtataagaaatactgtgagccaacaactttccatctcatgtcttctaaaatagaaatgagaatctttttctgaggtttccagagctcttgcaaaataatccaatcttaaaattctctgtatcttttaatgtactttaaactaatagaattcattcttgttgaatcttaagtttgccaaagttgcaagctaatattcattgttttccctgtctgcacccttcagtcagttggtcattcttaaattcatacacttgtaacacttggtgttatttcagaatatttagcatgataactgatcattatcatgatcattcattaaacctgtccttgactgacaacgcagaatagttgtgaatagttgtgatctgtcagatgtgctatgttgagttatagctctcctcaaaaaaaaaactgactttgtggaattgggagaggatggaacatttcttcatgcaactatgccctgttatcattgtacttttgtttattttgttttcacagtactgtgtttttcatgtgtgtgtttgttttgtgttgagcctcgtcgtggccgtagcatgtcggtttgtgttcccatctttctgctgttgcctctgtgtcccgcatctctcccagtgctcttccaccccaccgccagtactgtctgcacctctttctccttccctcctccggactgccccgaggaaacttttgccgaaaagctttctaaagcattggagagtgtcctgcctatgcactctgcctctcagcgcaagcaccgacgctccagcctgccttccctctttgtcagtactgtatgtaactgtaaacttctgacaaatgaacaattattaccaatgaatacatccaggcaacaagaattttaattaaaattgaataaagaacaggactaaacttggttatattatgcttctaggataccagttattcctgtaggaaacttttttgtaataaggctttaagggggaataaaaagcaaggaggtaatgagagagaatacctataatgtccttggcacataccagcatgtgtccaataaaatttacctttcattatatcttcctgtttcctatcgacataaaattctttcattttcatattctagttttaaaaggattaattttaaagttattgccttaatcaaaagcaaattttatcaaacattacccaaacattttttctcctcactttattctaaggagacactaataaaactggatgagtcctttttttttttaataaccttagtcagcaaagaataaccttggagtgaacatttattttaatttaatcatttatgtaagatgatgtatcagcaaaccaaagaactacataatgttcccccatggaagtattttttccttaggtttcaacctaaggtgggactgtagctctaatgaatgcattagatgcatatatagtatgtatgactttctttggctccattaatgtttataaaagatctatcttataaaattattttaagcatgtgggtccttgctgcttttccaaataaagaatatagttaactttctgaactagcaaccccagaattttcagatttttacttaggagaatgaagaactatacagtgttattatggctttgagacataccatgagtaaataacaataaaatgagcattctgactgaaaatagaaatccttatttagctattgtgaacagttgatctactttgccagctcgataaggatcttccctatggaagaatgaaactgaattcttttaaaaagcgggggaggctgtgatagtgaattcttactattatttacttatttatttatgttttggagatggaatctcgctctgtcgccaggctggagtgcagtggcacgatctcgactcacggcaacctctgcctcctgggttcaagtgattctcctgcctcagcctcccgagtagctaggactacaggcgcatgccaccacacccagctaatttttgtattttagtagagatgggtttcaccatgtttggccaggatggtcttgatctcctgacattgtgatccacccccttcagcctcccacagtgctgggattacaggcgtgagccaccgcgcccagcgggtaatgaattattatagtgtatattgtttgcatttggcaagctgtgtttctaaatttagtcttaaatctagaactgcttctagaataaaaagtgctatcaaataaaattggctgtcatttatattacccttttcagttgattagaacaccaggatgtcagaatcagaaaattcatggttacggccgggctcagtggctcacgcctgtaatcctagcactttgggaggccgaagtgagcagatcacctgaggtcaggagttcgagaccagcctggccaacatggcgaaatgtgtctctactaaaaatacaaaaattagctgggcgtggtggcgcacgcgtgtagtcccagctacttgggaggctgaggcagaagaatcccttgaacccaggaggcggaggttgcagtaagccaagattgtcactgcactccagcctgggcgaaagaggaagactccatctcaaaaaaaagaaaaaagaaatttcatggttatgcaactcttatttatgatcagaaaaatggacattttgtgatttaactctgtaacatgtttcatgtagtaaaaatataataaaactattaatcatctagcttgggagagataggagaaagacattactgtcactagtcaaattatatatcttttactatccaccaaaaatctcttctgatttctggttagaaggcatactattaattgataagaaaataaaactgaaggcctctaacatatcacagggtaataagaatatagggaaagttagttcaatagtttaaattaaagcacacttcttacagtatagaactagtcgggcttttatgccttgttttagttcttactcttcctttaactctttttctgttgatgtaatttacattaatgcttaagagtgaactttttaagtgtgggtaaaaacgaaataattacttacaaagtttaattcttccatttcctttgagagaggaaagttatggaaaagcagctcttatctaaagcaaagagcccacagattgatttcattggccctggatgtatttaatggatttttactatgcacataatttccagaagcattgttatttatttattaattataaatttagtgtaaccatttcatagggttacacagaactacccagttgtgcatgtctgatgtaatttcacatatgaatg

SEQ ID NO: 7 - DNA, 1755 bp, Mus musculus
tttgctttgacagagcaagtttgtgggaaagcaactggggtggtttttttttttttttttcattcaaggaagtgccacagattaactttattggccctggatgtatttaatggatttttgctataaattccaaaagcattgttatttattaaaattttcgtgtggccacttcatggggttacacagaactatccagctgtgcatgtctgatgtaattttacatattgaatgtatgaattacttgtcttattcatgttgatacagcctcagtccatggcgcatccgtgtggggggaccccaacatacccagaatcacagatatttttcccaactattcatgaacgtccagtttctttttcaccacctcccacctgtccaccgaaagtagccatttcccaacgacgtaagagcacctccttcctggaagcccaaactcgccacttccaacccctgctgaggactgttggccaaaatcatcttccacctggtagcagcccaactaactggacaccagaggccatagttatgttgggtgctacagccaatagagtaaatagagagctatgtgagatgcaggtccaacctgtgtttgagccaacccagatttacagtgactatagacctggactagtactggcagaagaagctcactattttattcctcaggaaacagtatatctagctggggtgcattaccaggcccaggtggcaggacagtatgagggcatttcatacaactcaccagtactgtcaagtcctatgaaacagatatctgaacagaagccagtgccggggggccctgcctcaagttctgtctttgaatttccttctggacaggctttcctggtaggacaccttcagaatttaagattagattctggaccaagtccagcatcacccctctctagtatttctgcgcctaacagtacagatgctacacatctgaaatttcaccctgtctttgtacctcattctgcaccagctgtgttaactaatagcaatgagaacagaagcaactgtgtatttgaatttcatgctcaaacaccaagttcttcaggagaaggaggtgggattttacctcaacgtgtttaccgaaatcggcaggttgcagtggactcaaatcaggaagaactgtctcctcagtcagttggattacattgccacctgcagcctgttactgaagaacagcgtaataaccatgcccccgaattgaccatttctgtggtagaacctatgggacaaatctggccaataggaagcccagaatattccagtgattcctctcaaattacttcttcagatctcagtgattttcaatcacctccccctacaggggggacagctgcaccttttggctctgacgtctcattgccctttattcgcctgcctcagacagtgttacaagaatccccactattcttctgtttcccccaaggaaccacatctcagcaggtcttatctgcttcatattcttcaggaggatctacactccatccacaggttataggaaaactttcacaattcttttaaactaccctattatacaccagtgcatttaaattctctatctcatttacctgaatcatggatttataaggaatgttgtttattttcctagtaggatttcttttgtggttataaatacttgtagtaaaaaatgaactataaagcagaaaatacctggtttgcctgtgattttaattgtcagaatcaatgactaatctctttggaattttgtggtagtaatgaaaatgc

SEQ ID NO: 8 - protein, 433 aa, Mus musculus
MYELLVLFMLIQPQSMAHPCGGTPTYPESQIFFPTIHERPVSFSPPPTCPPKVAISQRRKSTSFLEAQTRHFQPLLRTVGQNHLPPGSSPTNWTPEAIVMLGATANRVNRELCEMQVQPVFEPTQIYSDYRPGLVLAEEAHYFIPQETVYLAGVHYQAQVAGQYEGISYNSPVLSSPMKQISEQKPVPGGPASSSVFEFPSGQAFLVGHLQNLRLDSGPSPASPLSSISAPNSTDATHLKFHPVFVPHSAPAVLTNSNENRSNCVFEFHAQTPSSSGEGGGILPQRVYRNRQVAVDSNQEELSPQSVGLHCHLQPVTEEQRNNHAPELTISVVEPMGQIWPIGSPEYSSDSSQITSSDLSDFQSPPPTGGTAAPFGSDVSLPFIRLPQTVLQESPLFFCFPQGTTSQQVLSASYSSGGSTLHPQVIGKLSQFF

SEQ ID NO: 9 - DNA, 1716 bp, Rattus rattus
gcagctttttctttcattcaaggaagtgccacagattgactttattggccctggatgtatttaatggatttttgctataaattccaaaagcattgttagttattaaaattttggtgtagccacttcatggggttacacagaactatccagctgtgcatgtctgatgtaattttacatattgaatgtatgaattacttgtcttattcatgttgatacagcctcagtccgtggcgcatccgtgtggggggaccccaacatacccagaatcacagatatttttcccaactattcatgaacgtccagtttctttttcaccacctcctacctgtccaccgaaagtagccatttcccagcgacgtaagagtacctccttcctggaagctcaaactcgccacttccaacccctgctgaggactgttggccaaaatcatcttccacctggtggcagcccaactaactggacaccagaggccatagttatgttgggtactacagccaatagagtaaatagagagctatgtgagatgcaggtccaacctgtgtttgagacaacccagatttacagtgactatagacctggactagtactggcagaagaagctcgctattttatcctcagggaaacagtatatctaggtggggtgcattaccatccccatgcggcaggacagtatgagggtatttcatacaactcaccagtactgtcaagtcctatgaaacagataactgaacagaagccagtgcctgggtgccctgcctcaagttctgtctttgaatttccttctggacaggctttcctagtaggacaccttcagaatttaagattagattctggaccaagtccagcatcacccctgtctagtatttctgcgcctaacagtacagatgctacacatttgaaatttcaccctgtctttgtacctcattctgcaccagctgtgttaactcatagcaatgagaacagaagcaactgtgtatttgaatttcatgctcaaacaccaagttcctcttcaggagaaggaggtgggattttacctcaacgtgtttaccgaaatcgacaggttgcagtggactcaagtcaggaagaactgtctcctcagtcagttggattacactgccacctgcagcctgttactgaagaacagcgtaataaccataccccagaattgaccatttctgtggtagaacctatgggacaaaactggccagtaggaagcccagaatattccagtgattcctctcaaattacttcttcagatatcagtgattttcaatcacctccccctacagggggaacagctgcaccttttggctctgacgtctcattgccctatattcgcctgcctcagacagtgttacaagaatccccactattcttctgtttcccccaaggaaccacatctcagcaggtcttatctgcttcatattcttcaggaggatctgcactccatccacaggttataggaaaactttcacaattcttttaaaataccctattatgcaccaatgcatttaaattctctgtctcatttccctgaatcatggatttataagaaatgttattttcttagtaggatttcttttgtggttataaatatatgtactaaaaaaatgaactttaaagtggaaaatacctggtttacctgtgattttagttgtcagaatcaatgactaatatctgtggaattttgtggtagtagtaaaaatgcaattgtc

SEQ ID NO: 10 - protein, 434 aa, Rattus rattus
MYELLVLFMLIQPQSVAHPCGGTPTYPESQIFFPTIHERPVSFSPPPTCPPKVAISQRRKSTSFLEAQTRHFQPLLRTVGQNHLPPGGSPTNWTPEAIVMLGTTANRVNRELCEMQVQPVFETTQIYSDYRPGLVLAEEARYFILRETVYLGGVHYHPHAAGQYEGISYNSPVLSSPMKQITEQKPVPGCPASSSVFEFPSGQAFLVGHLQNLRLDSGPSPASPLSSISAPNSTDATHLKFHPVFVPHSAPAVLTHSNENRSNCVFEFHAQTPSSSSGEGGGILPQRVYRNRQVAVDSSQEELSPQSVGLHCHLQPVTEEQRNNHTPELTISVVEPMGQNWPVGSPEYSSDSSQITSSDISDFQSPPPTGGTAAPFGSDVSLPYIRLPQTVLQESPLFFCFPQGTTSQQVLSASYSSGGSALHPQVIGKLSQFF

SEQ ID NO: 11 - protein, 486 aa, Takifugu rubripes
SHFECHSIHCISLSQQHSVPHPYSGGAGGGAGGNIPLASLPDPTTVLFPSIPERPISFSPPPTMPPKAYNNQRRKSTSILEAHTRHFQPAYTRYGSNLHPFSGMETVEPPPLFMVNPGFAAAAQRLGVGEPLHLPGQSDPSLYGYKDMRAEHGEAMRRLSLNQAALLDHYEAMAYGGYPMTAHQLSRLSFHQQRHAAAAAASFAFEQPGFLHPHLIQRMSAHSPVPPPLAPLSAPSGTSMSASSEGCYPSPQHAAVSTFAISAPPVMPDAAPPTGNVFEFHLAAAGDPNLMASRLFRARRSSMDLPLEDNTGAGSGGSGTYVRLQPVTEELYSYVSPELPLPPGSLFLHHAGLTAKDKSPDPSSDSLASSDNGEFQSPPPPPLLPSFDSSSAQSIPHTASTAMYDPSTGMLPIDPQGHPPSMQSFLPPTPSSELPLGGAPSTQLETLMQSAWARHGGVVPAQPDMTYHESLLAMQAVNPSLVLLRF

SEQ ID NO: 12 - DNA, 13546 bp, Homo sapiens
ccctcaagtatctacaaagatggtagggaaatgcataatgcagattttacagttcggttaaggccagtcctgagagaattctttcttcctcctcttctctactttttttacaatgccttttttttttttggcggggggtggtggtggggggtgttgttttctttcaatatactactgcttaatttacccttttattctgtaggtagagtctgggtatgtctgtgaaggtgatcacaagaccatggctaaagctatcaaagacagagtatcattaattaagaggaaacgagagcagcggcagttggtacgggaggagcaagaaaaaaaaaagcaggaagagagcagtctcaaacagcaggtagaacaatccagtgcttcccagacaggaatcaagcagctcccttctgctagcaccggcatacctactgcttctaccacttcagcttcagtttctacacaagtagaacctgaagaacctgaggcagatcaacatcaacaactacagtaccagcaacccagtatatctgtgttatgtacgtatcttgggaagtggacagataggctaatgattctcctaattggtttttttagcttcttgttattttgcactggctttttggttttgaattatgaatcctactattatacaaaatttgaaatggttgtcgtctagcttctctttattcttatatctgtgactaggagaatagaaggatggggtggtaaggaaatgtgattaggatacaaaataattttataagcttcaacatcttggaggtttcaatttgtagatttctacttttcacaatttatttaaataaaacagatttaataatgaagaatgaacaagcttggctactaaaattgaacccatcagtctggttgagctcttttagcaaagctgaatggtatatagactaaaaactaatgagaacactggttattaatttaattacttgtttaatactgagaacctcctggcctccataattaagactttgaaagctgtgtcaaatttaaggcttatagtgtttcatgctgtttcccttactcctcaggtggtaagattctgtgatagccatatggattcctcatttaaatatagaaatctactgaatatggctacaattcattttttatttagttcaaatctgtgcaaggaataactagttacccctaatataatgggtctaaatatgagaaaatgtgaacctccattttgtgatttgtctttctctctctcttttttttggcgattcatttttccttcagctgatgggacggttgacagtggtcagggatcctctgtcttcacagaatctcgagtgagcagccaacagacagtttcatatggttcccaacatgaacaggcacattctacaggcacagtcccagggcatataccttctactgtccaagcacagtctcagccccatggggtatatccaccctcaagtgtggtaagtaaatgcttaagagcatgtaatactacaatgagagccaatggatagtctgctaaattaaaattctttgtacaaaccaagtaacagtatgagtctgaagtagaaaatataaaatgctgtgtctctaaagccttattatagagtaggatatagacattaatgtgtgggcattagctgaactgcctacatgaagcagctatgtgcaagatttagattagtctgcatttttactaaagggaacgctctctttagcacttaagatgttgaaactattcacagcaacaaaagctggacatacaaatgttttgcgggtggggcgagaagagtttttcaaaagcttgttaaaatgattactaagtatttcctgtaagattccctattgtacaaaacacaggtaagaccttcaaaccattatagaatgctgcatgtgagtactctagattgagggaatcctttatttgaatgactgtttaatttagcgcttgtgttgagttaatcatcagttgctgtgtgtgttcctgccttttaaagtctgtgttggacacaagttccttccccagtatatgttcttttgctttttccctttagttggtaactgttttattactgatttattttcagaatcaacttatagaaattatttcctgaaacattttatgcttgtatacacatacacacacacaattatcctgtgttttacattagacactcttgtttctcatatagagaatgttaaagtagatcatcagataataccccatttcttttgttgacctcagaggaaaataaaatacgtcatctgcctctgaaaggtattagaaaataagtaacttgtcactgtacagcacttgtttagtgagatctttaagcagacagattttccccttctcagaaagcttttaatctaaactttgtggaattcttgctcttctttaaatgaaaatctttctctgttaagaaaatacttctgcttgttcgattttttatcctacagcttggaataaaagatgttcatttgtttcagagtccgtttaatcctgctttggctaggtctcaaaatgaatggccacagtaacacaaataaaaaactaatatactaactttttaaaacaatctactttctgtctttaaaataattgagggtagaatagaagaactttataaagactgcacagaaaatatatttctgtgttctgacaaatattacttgtcagcatttcttgtcagtttctacctttaattgttttttttctggattattataactagattttgctaagactttttttttaaaattttctcttgtctatactcatcatgggggagcattaggtctctattttagaaagcaagtagaatgacatgaagatattatttgagccattaatctagctatatttattgatgctctaaaagcagacgttcttgctttttaatgacagtattctatggaggtattatgaagaattattaaatattgagaatttaaaaatttgtctaattctattcttgtctcatttcaggaatgtggcttacaataatatggacaaattataagtgaaattttagaaattagaataaaagaaataaggctagagagaaatcaaaacaggaatgaaactaaacaatcatatgatgatatgtaaatacttggtattggtgggccacagatttggcccaaagcattctacacagccaagttttggaaatgaaaacatagtttcatagttcacaacatctaaaaggaaaataacagtcttattcaagacaacctttatggtattgatcatactgccttatagccatattaaaccctgtgcctgaacatttttttaagtttttttttttttttttgacagcgtctcactctgtctcccaggtgggaatacagtggcgtgatcttggctcactgcaactttcacctcctgggttcaagtgattctcctgcctcagcctccccagtagctaagattacaggcacccatcaccacgcccagctaatttttgtattttttttagtagagacacagtttcaccgtgttggctaggctggtcttgaactcttgacctcaagtgatccacctgcctcggcctcccaaagtgccaggattacaggtgtgagccaccatgcccggcctaaattattatttagagattcagattgaagatattctcccattgattactttgtaaaagtacaaaatatttcctatatccagttgtgaatttcttctagaaagctaaactttttcctagtagatcatgacttttttacctttacaaataaagagtggcaagtataagaaatactgtgagccaacaactttccatctcatgtcttctaaaatagaaatgagaatctttttctgaggtttccagagctcttgcaaaataatccaatcttaaaattctctgtatcttttaatgtactttaaactaatagaattcattcttgttgaatcttaagtttgccaaagttgcaagctaatattcattgttttccctgtctgcacccttcagtcagttggtcattcttaaattcatacacttgtaacacttggtgttatttcagaatatttagcatgataactgatcattatcatgatcattcattaaacctgtccttgactgacaacgcagaatagttgtgaatagttgtgatctgtcagatgtgctatgttgagttatagctctcctcaaaaaaaaaactgactttgtggaattgggagaggatggaacatttcttcatgcaactatgccctgttatcattgtacttttgtttattttgttttcacagtactgtgtttttcatgtgtgtgtttgttttgtgttgagcctcgtcgtggccgtagcatgtcggtttgtgttcccatctttctgctgttgcctctgtgtcccgcatctctcccagtgctcttccaccccaccgccagtactgtctgcacctctttctccttccctcctccggactgccccgaggaaacttttgccgaaaagctttctaaagcattggagagtgtcctgcctatgcactctgcctctcagcgcaagcaccgacgctccagcctgccttccctctttgtcagtactgtatgtaactgtaaacttctgacaaatgaacaattattaccaatgaatacatccaggcaacaagaattttaattaaaattgaataaagaacaggactaaacttggttatattatgcttctaggataccagttattcctgtaggaaacttttttgtaataaggctttaagggggaataaaaagcaaggaggtaatgagagagaatacctataatgtccttggcacataccagcatgtgtccaataaaatttacctttcattatatcttcctgtttcctatcgacataaaattctttcattttcatattctagttttaaaaggattaattttaaagttattgccttaatcaaaagcaaattttatcaaacattacccaaacattttttctcctcactttattctaaggagacactaataaaactggatgagtcctttttttttttaataaccttagtcagcaaagaataaccttggagtgaacatttattttaatttaatcatttatgtaagatgatgtatcagcaaaccaaagaactacataatgttcccccatggaagtattttttccttaggtttcaacctaaggtgggactgtagctctaatgaatgcattagatgcatatatagtatgtatgactttctttggctccattaatgtttataaaagatctatcttataaaattattttaagcatgtgggtccttgctgcttttccaaataaagaatatagttaactttctgaactagcaaccccagaattttcagatttttacttaggagaatgaagaactatacagtgttattatggctttgagacataccatgagtaaataacaataaaatgagcattctgactgaaaatagaaatccttatttagctattgtgaacagttgatctactttgccagctcgataaggatcttccctatggaagaatgaaactgaattcttttaaaaagcgggggaggctgtgatagtgaattcttactattatttacttatttatttatgttttggagatggaatctcgctctgtcgccaggctggagtgcagtggcacgatctcgactcacggcaacctctgcctcctgggttcaagtgattctcctgcctcagcctcccgagtagctaggactacaggcgcatgccaccacacccagctaatttttgtattttagtagagatgggtttcaccatgtttggccaggatggtcttgatctcctgacattgtgatccacccccttcagcctcccacagtgctgggattacaggcgtgagccaccgcgcccagcgggtaatgaattattatagtgtatattgtttgcatttggcaagctgtgtttctaaatttagtcttaaatctagaactgcttctagaataaaaagtgctatcaaataaaattggctgtcatttatattacccttttcagttgattagaacaccaggatgtcagaatcagaaaattcatggttacggccgggctcagtggctcacgcctgtaatcctagcactttgggaggccgaagtgagcagatcacctgaggtcaggagttcgagaccagcctggccaacatggcgaaatgtgtctctactaaaaatacaaaaattagctgggcgtggtggcgcacgcgtgtagtcccagctacttgggaggctgaggcagaagaatcccttgaacccaggaggcggaggttgcagtaagccaagattgtcactgcactccagcctgggcgaaagaggaagactccatctcaaaaaaaagaaaaaagaaatttcatggttatgcaactcttatttatgatcagaaaaatggacattttgtgatttaactctgtaacatgtttcatgtagtaaaaatataataaaactattaatcatctagcttgggagagataggagaaagacattactgtcactagtcaaattatatatcttttactatccaccaaaaatctcttctgatttctggttagaaggcatactattaattgataagaaaataaaactgaaggcctctaacatatcacagggtaataagaatatagggaaagttagttcaatagtttaaattaaagcacacttcttacagtatagaactagtcgggcttttatgccttgttttagttcttactcttcctttaactctttttctgttgatgtaatttacattaatgcttaagagtgaactttttaagtgtgggtaaaaacgaaataattacttacaaagtttaattcttccatttcctttgagagaggaaagttatggaaaagcagctcttatctaaagcaaagagcccacagattgatttcattggccctggatgtatttaatggatttttactatgcacataatttccagaagcattgttatttatttattaattataaatttagtgtaaccatttcatagggttacacagaactacccagttgtgcatgtctgatgtaatttcacatatgaatgtatgaattacttgtcttattcatgttgatacagcctcagtccatggcgcatccgtgtggggggaccccaacatacccagaatcacagatatttttcccaactattcatgaacgtccagtttctttttcaccacctcccacctgcccaccgaaagtagccatttcccagcggcgtaagagcacctccttcctggaagcccaaactcaccacttccaacccctgctgaggactgttggccaaagtcttcttccacctggtggcagcccaactaactggacaccagaggccgtagttatgttgggtactacagccagtagagtaactggagagtcatgtgagatacaggtccatcctatgtttgaaccatctcaagtttacagtgactatagacctggactagtacttccagaagaagctcactattttattcctcaggaagcagtgtatgtagctggggtacattaccaggcccgggtggcagaacagtatgagggcattccatacaactcatcagtactgtcaagtcctatgaaacagatacctgaacagaagccagtacaagggggccctacttcaagttctgtctttgaatttccatctggacaggctttcctggtaggacaccttcagaatctaagattagattctggattgggtccgggatctcccctctctagtatttctgcacctatcagtacagatgctacacgtttgaaatttcaccctgtctttgttcctcattctgcgcctgctgtgttaactcataacaatgagagcagaagcaactgtgtatttgaatttcatgttcacacaccaagctcctcttcaggagaaggaggtggaattttacctcagcgtgtttaccgaaatcggcaggttgcagtggacttgaatcaagaagaactgcctcctcaatcagttggattacatggctacttgcagcctgtgactgaagaaaagcataattaccatgccccagaattgaccgtttctgtggtagagcctatcggacagaactggccaataggaagcccagaatattccagtgattcctcacaaatcacttcttcagaccccagtgattttcagtcacctccccctacagggggagcagctgcaccttttggctctgacgtctcaatgccctttatccatctgcctcagacagtgttacaagaatccccacttttcttctgtttcccccaaggaaccacatctcagcaggtcttaactgcctcattttcttcaggaggatctgcacttcatccacaggttataggaaaacttccacaattattttaaactaccctactttgcaccataacatttaaattttctattccttatttccctgaatcatggattttggagaaatattgtttaattttatcagtagagtttccccatctttggggggttgtgaactacatatatgcatttaaaaacaaaatgtgagagaagctacctgatttacctattatatgtgaaaaccagtggaaaaaacacaaaaactagaattttagtcattcttcacaatcacgacttctatgcacgttattttcaaccagtagtgaaaatgcaagtgtatgtaatgtatggttgacccagcattatttaggaatacaaatcttaagtattactttcttcctccaaacaagtttttaaaaaataggataaattttttttctataaaatataaaacatggaaaatagggaatgctgtttttgaggtaatattaataatacacagaattttcattagtgtcgaaggatctaaaaagacaaagtatatcatgggaataaaaaaagatagaaaaggaaacagtttaggaatttgccttaacaaatgaaaatgcctttttaaaatggcatcagtcaagcaagttgctgtgcattattatatgtccaaataaaatgctaattcataaaattaaggactgattctaatttttagcaactaacaatctagtgagtggtgggctttttttttttgttataagaatttgtgatatgttgcaagctaaggaacagtagacatcttaggccttgtattttcctcaacaaatttaattttagttatttatgtgtatgagcatatacacacttagcaaataaagacaatgtaatgaatgtaatagaattcaaaagtacagtcagtgtttactgtttcatatattcatttggtatattttatcaaatgtgtacatcttataaaatctacatagaagactatgcagttatatattgaagacatttggatatcctacataaagtactatgtgttgttcttcctactttcttcctatgttcttccctcatcaggagcataaacaactcccaaaatgtgcacaaattatcaattatttagtgctgtactaagacacaaaaagatccagaagcttgacagctatacatgggcatgcagtatattcacatcagtattcaagttgcagcccctattataaatattaatgtattgccagtaattatcctttagactttccaaagtaaaaaataccaggtagttaatttatagacatactacagaaggaatcaatatttataatagttaagctgcagtggaatgtgatattatatccatgtaagctttaaggaaccccttgtaaactgaaatggaatgacaaaagtggtagccttttcatttcgttaacatacctatatatagtcctgcatcatttctcactttctagtgagaaacactgtagttggggtttctcagactcaacactgttgacatctgaggttggataattctttgttgtggtgggctgttctatgtattgtaggctatttagaacatccttggtttatacctcctaaatactagtagcagcaatctccctcagttgtgacaaccagaaatgtctccacatattgccaagtgcccccctgggggtggcaggattgctcctggatagcaatgatcgccctaggtacttggcagttgatgataacagattttttgatacttttgataaaagtcattgtggcactacaagaagtattaccaaattagcattaccaaatacctttctttaattcttcagataaatttcagattgatagtattaatactttgttcctgattattgtatttctaataacaaattttattatgaatctgacgattctagccagctaaaattacagcctgagcagcattgacagcttaggttttcactgtcagtgggttttgcatgggttgggatcagaacatgttgtgtggtatgatccttttctaaatttttcttacaaaagttgacccagaggcctctcccatacataatcaggttaatgtttcattttgatcaagcaaactctgaggagattaaatattcattgcaaaagcctgacctctatacacttactttaggccttctctaatttgttgtgttcacttctttccttcaggcacaggggcagagccagggtcagccatcctcaagtagcttaacaggggtttcatcttcccaacccatacaacatcctcagcaggtgagaacaatgcattgcaacattttatgaattagcagtggccagaacactattttaacttttgtgatttgttatataaatggcctgctaagtttttgaaaggaaaattaagaggcataccatgtcttgtgttcagtaacaaagaaaaaaaagagacttttctgtgtttcagaaaaatctcttttgtttaatatacttttgaacaatttagtagacatacattattactgatataattatctttttaaaagaggattctacaccttttttttgagacaaagtctcactctgtcacccaggctggagtgcagtggtgcaatcttagctcactgcaaccaccgcctcccaggttcaagcagtcctcctgcctcagctttccacgtagctgggactacaggtgcctgccaccacactcagctaatttttgtatttttagtagagacggggtttcaccatgctggccaggctggtctcaaactcctgacctcaggtaatccaccctcctcggcttcccaaagtgctgggattacaggcatgcgccacctcacccagcctggattctacacataatttacggtacaaagagtatgtcattccttttctctttttttccactgcttgcagcagtgatttgtcattaccttcattgtgcttgcctctactaatcctgcacttctttttaattatttctaatctgtagaatgatgtgtcagattttacgctaagaagcatctaatgaaaacctaagaacaattacttcaagaacatttatttatttatttatttttatttattttgagacagccttgctctgttgcccaggctggagtgcagtggtgcaatcttggctcactgcaacttccatctcccaggttcaagctattctcacgcctcagccgcccgagtagctgggactacaggcgcgtgccactatgcccagctaatttttgtattacgttctacttatgcctggctaatttagtagagatggcgtttcaccatgttggccaggctggtcttgaactcctgacctcaagtgatccacccgctttggcctcccaaagtgctgggagccactgtgcccagccaaaatattttaacttttgttttggggggcttgttccagaaagttcaaggaattcaagagattagtgtcatcctgatacatccaacagtaaaactggtaattctaaaattaaatatttatagtgcatcactgatattagttaatagtaattatttgcaagggaataatgcatttttggcataattgaatacttattagctattctacataaaatttgtagccactatgtatgtaatgcaaacttcctaatgttagttccccatgtgggttttttaatcagcaaactctaaactggcttctgtttctattagtttttggttttgaaacaaagttttcggttcttaattacaaatggccctttttcagtccaacatatggtcatgtagtcattcgatactggtttaagatcaggagttgcttgtacaagatgaaaatcacttaaaggcactttctacagaaacagttgattagtttgtttatacatgcattcactccgtgaactgaaggactgggacttatatgctttgttttgcacctgtcctttcttccaaacaagactggttaaattcaagtaaacatatttttgccaaccttaacaaaatctgttttattctattaacagttcctaattagaatgcaaattacaagtcacataaagctggttgagaatggaagtactgagtactaatctcctaacttgcttatctttggatgtagacgaacaccaatgaactgagcaaattgtatgtgctctgttttttcttttccaaaggaattggttatctgtcttgtatttggggatatgtatgtatacttactatcaaatgaagaagataataaaataattcattgagtttcatgttaagaaagagtgtgagaaaagaagagagcagtaccataaatgtcagattaaaagaattaggtagctacctctccaggagaaaaaaatatggctaaatgctatctctacagcagaaacaaaaattaatttttgtttcagtgatgtttttgttttagatacttttatagcatgtgttgtagaatcacaaaattgagccctcgatatatataggaagtgtattcggtctagaataattaacttctccaaagtcacttagataataagggagctacaaattataacccagaattcaagattcctagaatagtattgtttcttccattttaaataaagttctgcagggacttaatgattctgcaaatgtacctcagaagctgtctttatttgttttaattattggagttccacgtttgaaaaaagaaaggttgaaaactttttgtactatatggatagtctatttttagacatgatttttgttttacgtactttcaggtcccaatcccaccttagagaaccatatctcttgttttattttagtgaggaaagaaatatacttttgctaaaatttactttatttcatgcgtttcttgaacatagtaaatgaggaattgcttttttgtgtgtttttgtagggtctcgtatagtgatatactagatcttagcaaggtaccaaaaatagctgcctcctgctgaaaagctttgtgaccacaaacaaagttgtgaactttgtcagacacagtgccagctacttggaaggctgaggcaggaagatcacttgagcccaggagttcaaggctacagtgaactatgatattgccaccatactccagcctgggcaacagagcatgactcatctcaaaaaaaaaaaagcaccgaagttgtgaactacctaccctactatccataaagtctgtttgaactggtttcatgaggcaagcttgggcagagcatgatgctaaactgttaagctagaccacccctgcctggctgaagtgctgattattagtcagtgggactgcgcacagcagatcatcatgtacttctggcatatccagaggt

SEQ ID NO: 13 - protein, 509 aa, Tetraodon nigroviridis
  Partial amino acid sequence
LCHALSFISSGLCWGFSLFHSECHSEIHCISLSQQHSMPHPYSGGAGGGAGGNVPLASLPDPTAVLFPSIPERPISFSPPPTMPPKAYNTQRRKSTSILEAHTRHFQPAYPRYGSNLHPFSGMEAVEPPPLFMVNPGFPSAAQRLGVGEPLHLPGQSDPSLYGYKDMRAEHGEAVRRLSQAALLDHYEAMAYGGYPMAAHQLGRLTFHQQRHAAAAAAAAAAAASFGFDQPGFLHPHLVQRMSAHSPVPPPLAPLSAPSGTSISSSSEGCYPPPHAAASPFAVSAPPVMADSAAPPTGNVFEFHLAAAGDPNLMASRLFRARRSSXXXXXXXXXXXXXXXXXXXXXXXXXXXXXXXXXXXXXXXXXXXXXXXXXXXXXXXXXXXXXXXXXXXXXXXXFQSPPPPPLLPSFDSSCAQSIPHSTSTATYDPPGGMFPIDPQGHPPSMQSFLPPTPSSELPLGGAPSTHLETLMQSAWARHGGVVPAQPDMTYHESLLAMQAVKPSLVLLPF

SEQ ID NO: 14 - DNA, 2785 bp, Homo sapiens
cgtggtggcgcacgcgtgtagtcccagctacttgggaggctgaggcagaagaatcccttgaacccaggaggcggaggttgcagtaagccaagattgtcactgcactccagcctgggcgaaagaggaagactccatctcaaaaaaaagaaaaaagaaatttcatggttatgcaactcttatttatgatcagaaaaatggacattttgtgatttaactctgtaacatgtttcatgtagtaaaaatataataaaactattaatcatctagcttgggagagataggagaaagacattactgtcactagtcaaattatatatcttttactatccaccaaaaatctcttctgatttctggttagaaggcatactattaattgataagaaaataaaactgaaggcctctaacatatcacagggtaataagaatatagggaaagttagttcaatagtttaaattaaagcacacttcttacagtatagaactagtcgggcttttatgccttgttttagttcttactcttcctttaactctttttctgttgatgtaatttacattaatgcttaagagtgaactttttaagtgtgggtaaaaacgaaataattacttacaaagtttaattcttccatttcctttgagagaggaaagttatggaaaagcagctcttatctaaagcaaagagcccacagattgatttcattggccctggatgtatttaatggatttttactatgcacataatttccagaagcattgttatttatttattaattataaatttagtgtaaccatttcatagggttacacagaactacccagttgtgcatgtctgatgtaatttcacatatgaatgtatgaattacttgtcttattcatgttgatacagcctcagtccatggcgcatccgtgtggggggaccccaacatacccagaatcacagatatttttcccaactattcatgaacgtccagtttctttttcaccacctcccacctgcccaccgaaagtagccatttcccagcggcgtaagagcacctccttcctggaagcccaaactcaccacttccaacccctgctgaggactgttggccaaagtcttcttccacctggtggcagcccaactaactggacaccagaggccgtagttatgttgggtactacagccagtagagtaactggagagtcatgtgagatacaggtccatcctatgtttgaaccatctcaagtttacagtgactatagacctggactagtacttccagaagaagctcactattttattcctcaggaagcagtgtatgtagctggggtacattaccaggcccgggtggcagaacagtatgagggcattccatacaactcatcagtactgtcaagtcctatgaaacagatacctgaacagaagccagtacaagggggccctacttcaagttctgtctttgatttccatctggacaggctttcctggtaggacaccttcagaatctaagattagattctggattgggtccgggatctcccctctctagtatttctgcacctatcagtacagatgctacacgtttgaaatttcaccctgtctttgttcctcattctgcgcctgctgtgttaactcataacaatgagagcagaagcaactgtgtatttgaatttcatgttcacacaccaagctcctcttcaggagaaggaggtggaattttacctcagcgtgtttaccgaaatcggcaggttgcagtggacttgaatcaagaagaactgcctcctcaatcagttggattacatggctacttgcagcctgtgactgaagaaaagcataattaccatgccccagaattgaccgtttctgtggtagagcctatcggacagaactggccaataggaagcccagaatattccagtgattcctcacaaatcacttcttcagaccccagtgattttcagtcacctccccctacagggggagcagctgcaccttttggctctgacgtctcaatgccctttatccatctgcctcagacagtgttacaagaatccccacttttcttctgtttcccccaaggaaccacatctcagcaggtcttaactgcctcattttcttcaggaggatctgcacttcatccacaggttataggaaaacttccacaattattttaaactaccctactttgcaccataacatttaaattttctattccttatttccctgaatcatggattttggagaaatattgtttaattttatcagtagagtttccccatctttggggggttgtgaactacatatatgcatttaaaaacaaaatgtgagagaagctacctgatttacctattatatgtgaaaaccagtggaaaaaacacaaaaactagaattttagtcattcttcacaatcacgacttctatgcacgttattttcaaccagtagtgaaaatgcaagtgtatgtaatgtatggttgacccagcattatttaggaatacaaatcttaagtattactttcttcctccaaacaagtttttaaaaaataggataaattttttttctataaaatataaaacatggaaaatagggaatgctgtttttgaggtaatattaataatacacagaattttcattagtgtcgaaggatctaaaaagacaaagtatatcatgggaataaaaaaagatagaaaaggaaacagtttaggaatttgccttaacaaatgaaaatgcctttttaaaatggcatcagtcaagcaagttgctgtgcattattatatgtccaaataaaatgctaattcataaaattaagg

SEQ ID NO: 15 - DNA, 2787 bp, Homo sapiens
cgtggtggcgcacgcgtgtagtcccagctacttgggaggctgaggcagaagaatcccttgaacccaggaggcggaggttgcagtaagccaagattgtcactgcactccagcctgggcgaaagaggaagactccatctcaaaaaaaagaaaaaagaaatttcatggttatgcaactcttatttatgatcagaaaaatggacattttgtgatttaactctgtaacatgtttcatgtagtaaaaatataataaaactattaatcatctagcttgggagagataggagaaagacattactgtcactagtcaaattatatatcttttactatccaccaaaaatctcttctgatttctggttagaaggcatactattaattgataagaaaataaaactgaaggcctctaacatatcacagggtaataagaatatagggaaagttagttcaatagtttaaattaaagcacacttcttacagtatagaactagtcgggcttttatgccttgttttagttcttactcttcctttaactctttttctgttgatgtaatttacattaatgcttaagagtgaactttttaagtgtgggtaaaaacgaaataattacttacaaagtttaattcttccatttcctttgagagaggaaagttatggaaaagcagctcttatctaaagcaaagagcccacagattgatttcattggccctggatgtatttaatggatttttactatgcacataatttccagaagcattgttatttatttattaattataaatttagtgtaaccatttcatagggttacacagaactacccagttgtgcatgtctgatgtaatttcacatatgaatgtatgaattacttgtcttattcatgttgatacagcctcagtccatggcgcatccgtgtggggggaccccaacatacccagaatcacagatatttttcccaactattcatgaacgtccagtttctttttcaccacctcccacctgcccaccgaaagtagccatttcccagcggcgtaagagcacctccttcctggaagcccaaactcaccacttccaacccctgctgaggactgttggccaaagtcttcttccacctggtggcagcccaactaactggacaccagaggccgtagttatgttgggtactacagccagtagagtaactggagagtcatgtgagatacaggtccatcctatgtttgaaccatctcaagtttacagtgactatagacctggactagtacttccagaagaagctcactattttattcctcaggaagcagtgtatgtagctggggtacattaccaggcccgggtggcagaacagtatgagggcattccatacaactcatcagtactgtcaagtcctatgaaacagatacctgaacagaagccagtacaagggggccctacttcaagttctgtctttgaatttccatctggacaggctttcctggtaggacaccttcagaatctaagattagattctggattgggtccgggatctcccctctctagtatttctgcacctatcagtacagatgctacacgtttgaaatttcaccctgtctttgttcctcattctgcgcctgctgtgttaactcataacaatgagagcagaagcaactgtgtatttgaatttcatgttcacacaccaagctcctcttcaggagaaggaggtggaattttacctcagcgtgtttaccgaaatcggcaggttgcagtggacttgaatcaagaagaactgcctcctcaaatcagttggattacatggctacttgcagcctgtgactgaagaaaagcataattaccatgccccagaattgaccgtttctgtggtagagcctatcggacagaactggccaataggaagcccagaatattccagtgattcctcacaaatcacttcttcagaccccagtgattttcagtcacctccccctacagggggagcagctgcaccttttggctctgacgtctcaatgccctttatccatctgcctcagacagtgttacaagaatccccacttttcttctgtttcccccaaggaaccacatctcagcaggtcttaactgcctcattttcttcaggaggatctgcacttcatccacaggttataggaaaacttccacaattattttaaactaccctactttgcaccataacatttaaattttctattccttatttccctgaatcatggattttggagaaatattgtttaattttatcagtagagtttccccatctttggggggttgtgaactacatatatgcatttaaaaacaaaatgtgagagaagctacctgatttacctattatatgtgaaaaccagtggaaaaaacacaaaaactagaattttagtcattcttcacaatcacgacttctatgcacgttattttcaaccagtagtgaaaatgcaagtgtatgtaatgtatggttgacccagcattatttaggaatacaaatcttaagtattactttcttcctccaaacaagtttttaaaaaataggataaattttttttctataaaatataaaacatggaaaatagggaatgctgtttttgaggtaatattaataatacacagaattttcattagtgtcgaaggatctaaaaagacaaagtatatcatgggaataaaaaaagatagaaaaggaaacagtttaggaatttgccttaacaaatgaaaatgcctttttaaaatggcatcagtcaagcaagttgctgtgcattattatatgtccaaataaaatgctaattcataaaattaagg

SEQ ID NO: 16 - DNA, 2786 bp, Homo sapiens
cgtggtggcgcacgcgtgtagtcccagctacttgggaggctgaggcagaagaatcccttgaacccaggaggcggaggttgcagtaagccaagattgtcactgcactccagcctgggcgaaagaggaagactccatctcaaaaaaaagaaaaaagaaatttcatggttatgcaactcttatttatgatcagaaaaatggacattttgtgatttaactctgtaacatgtttcatgtagtaaaaatataataaaactattaatcatctagcttgggagagataggagaaagacattactgtcactagtcaaattatatatcttttactatccaccaaaaatctcttctgatttctggttagaaggcatactattaattgataagaaaataaaactgaaggcctctaacatatcacagggtaataagaatatagggaaagttagttcaatagtttaaattaaagcacacttcttacagtatagaactagtcgggcttttatgccttgttttagttcttactcttcctttaactctttttctgttgatgtaatttacattaatgcttaagagtgaactttttaagtgtgggtaaaaacgaaataattacttacaaagtttaattcttccatttcctttgagagaggaaagttatggaaaagcagctcttatctaaagcaaagagcccacagattgatttcattggccctggatgtatttaatggatttttactatgcacataatttccagaagcattgttatttatttattaattataaatttagtgtaaccatttcatagggttacacagaactacccagttgtgcatgtctgatgtaatttcacatatgaatgtatgaattacttgtcttattcatgttgatacagcctcagtccatggcgcatccgtgtggggggaccccaacatacccagaatcacagatatttttcccaactattcatgaacgtccagtttctttttcaccacctcccacctgcccaccgaaagtagccatttcccagcggcgtaagagcacctccttcctggaagcccaaactcaccacttccaacccctgctgaggactgttggccaaagtcttcttccacctggtggcagcccaactaactggacaccagaggccgtagttatgttgggtactacagccagtagagtaactggagagtcatgtgagatacaggtccatcctatgtttgaaccatctcaagtttacagtgactatagacctggactagtacttccagaagaagctcactattttattcctcaggaagcagtgtatgtagctggggtacattaccaggcccgggtggcagaacagtatgagggcattccatacaactcatcagtactgtcaagtcctatgaaacagatacctgaacagaagccagtacaagggggccctacttcaagttctgtctttgaatttccatctggacaggctttcctggtaggacaccttcagaatctaagattagattctggattgggtccgggatctcccctctctagtatttctgcacctatcagtacagatgctacacgtttgaaatttcaccctgtctttgttcctcattctgcgcctgctgtgttaactcataacaatgagagcagaagcaactgtgtatttgaatttcatgttcacacaccaagctcctcttcaggagaaggaggtggaattttacctcagcgtgtttaccgaaatcggcaggttgcagtggacttgaatcaagaagaactgcctcctcaatcagttggattacatggctacttgtagcctgtgactgaagaaaagcataattaccatgccccagaattgaccgtttctgtggtagagcctatcggacagaactggccaataggaagcccagaatattccagtgattcctcacaaatcacttcttcagaccccagtgattttcagtcacctccccctacagggggagcagctgcaccttttggctctgacgtctcaatgccctttatccatctgcctcagacagtgttacaagaatccccacttttcttctgtttcccccaaggaaccacatctcagcaggtcttaactgcctcattttcttcaggaggatctgcacttcatccacaggttataggaaaacttccacaattattttaaactaccctactttgcaccataacatttaaattttctattccttatttccctgaatcatggattttggagaaatattgtttaattttatcagtagagtttccccatctttggggggttgtgaactacatatatgcatttaaaaacaaaatgtgagagaagctacctgatttacctattatatgtgaaaaccagtggaaaaaacacaaaaactagaattttagtcattcttcacaatcacgacttctatgcacgttattttcaaccagtagtgaaaatgcaagtgtatgtaatgtatggttgacccagcattatttaggaatacaaatcttaagtattactttcttcctccaaacaagtttttaaaaaataggataaattttttttctataaaatataaaacatggaaaatagggaatgctgtttttgaggtaatattaataatacacagaattttcattagtgtcgaaggatctaaaaagacaaagtatatcatgggaataaaaaaagatagaaaaggaaacagtttaggaatttgccttaacaaatgaaaatgcctttttaaaatggcatcagtcaagcaagttgctgtgcattattatatgtccaaataaaatgctaattcataaaattaagg

SEQ ID NO: 17 - DNA, 2365 bp, Sus scrofa
gaattcggcacgagggtatccaccaaaaatctcttctgatttctaggtagaagacacactattagttaatcagaaattaactctggagcctctaacagcacacaacaataaagcttcagaataggaaaacttggtacagtctcattcttacttaactctgtttccactgatgcagtttacattaagtcatgagtgaacatattaattgtgggtaaaagcagaataatcacttaggaagttaaatccttccatttcccttgagagaggagaattgtggaaaagcagctcctatctaatgcaaagagtcccacagaataattttattgaccctggatgtatttaatggattttttttttttactgtgcacataatttctgaaagcattgttatttctttattaattataaatttggtgtaaccatttcgtagggttacacagagctacccagttatgcatgtctaatgtaatttcacatatgaatgtatgaattacttgtcttattcatgttgatacagcctcagtccatggcgcatccgcgtggggggaccccaacatacccagaatcacagatatttttcccaactattcatgaacgtccagtttctttttcaccacctcccacctgtccaccgaaggtggctatttcccagcggcgtaagagcacctccttcctggaagcccaaactcatcacttccaacccctgctgaggactgttggccaaaatcttcttccacctggtggctgcccaactaactggacaccagaggccgtagttatgttgggtactacagccagtagagtaactggagagccatgtgagatacaggtccaacctctgtttgagcctactcaagtttatggtgactgtagacctggactagtacttccagaagaagctcactactttattcctcaggaagcagtgtatgtagcaggggtacattaccagacccaaatggcagaacagtttgagggtattccatacaactctccagtcctgtcaagtcctatgaaacagatacctgaacagaagccagtgcaagggggccctccttcgagttctgtctttgaatttccatctggacaggctttcctggtaggacatcttcagaatttacgattagattctggactaagtccaggatctcccctctctagcatttctacacctatcagtacagatgctacacgtttgaaatttcaccctgtctttgttcctcattctgcacctgctgtgttaactcataacaatgagagcagaagcaattgtgtatttgagtttcatgttcatactccaagctcctcttcaggagaaggaggagttttacctcagcgtatttaccgaaatcgacaggttgccgtggacttgaatcaggaagaaccacctcctcaatcagctggattacacggccgcctgcagcctgtgactgaagaacaacataatttccagcccccagaattgaccgtttctgtggtagagcctactggacagagctggccaataggaagcccagaatattccagtgattcctcacaaatcacttcttcagaccccagtgattttcaatcacctccccctacagggggaacagctgcaccttttggctctgacgtctcattaccctttatccatctgcctcagacagtgatacaagaatccccacttttcttctgtttcccccaaggaaccacttctccgcagattttatctgcatcattttcttcaggaggatctgcactccatccacaggttataggaaaacttccacagttctcttaaactaccctgctgtgcaccattgcatttaagttttctgtccctcctttccattcatgaggattattgtttaattttattacaatttctccattttgtgtgtgtgttgggggggttattttaaactacttgtatttaagaacaaaatctgagagcaagcctgcctgatttacctataatcaaaccattggaaaagaaaagaaactagaattttagttacttgtcaaaatcagtgacttctgttcatatgtagaacgttttcaactagaagtgaaatgtaaatgtctgtaaagttgactcagaattgatgcttcacacatactaatgtgaagtatttctttcttcctagaaatgaacgtggctagttttttaagagaagataacatttttttttcctgtaaaatgaaaagaatgggaagtggagaatcctttttctaaaagtagtattaggagtacatacagaaattaaaagagaggaaacatcttaataagtgaagttgcctttttaaaatggcatcaaacaaatcaatttgtattatgtgttcaaataaaatgttaatccataaaaaaaaaaaaaaaaaaaaa

SEQ ID NO: 18 - protein, 433 aa, Sus scrofa
MYELLVLFMLIQPQSMAHPRGGTPTYPESQIFFPTIHERPVSFSPPPTCPPKVAISQRRKSTSFLEAQTHHFQPLLRTVGQNLLPPGGCPTNWTPEAVVMLGTTASRVTGEPCEIQVQPLFEPTQVYGDCRPGLVLPEEAHYFIPQEAVYVAGVHYQTQMAEQFEGIPYNSPVLSSPMKQIPEQKPVQGGPPSSSVFEFPSGQAFLVGHLQNLRLDSGLSPGSPLSSISTPISTDATRLKFHPVFVPHSAPAVLTHNNESRSNCVFEFHVHTPSSSSGEGGVLPQRIYRNRQVAVDLNQEEPPPQSAGLHGRLQPVTEEQHNFQPPELTVSVVEPTGQSWPIGSPEYSSDSSQITSSDPSDFQSPPPTGGTAAPFGSDVSLPFIHLPQTVIQESPLFFCFPQGTTSPQILSASFSSGGSALHPQVIGKLPQFS

SEQ ID NO: 19 - protein, 426 aa, Homo sapiens [sequence omitted]

SEQ ID NO: 20 - protein, 419 aa, Homo sapiens [sequence omitted]

SEQ ID NO: 21 - protein, 425 aa, Mus musculus
MLIQPQSMAHPCGGTPTYPESQIFFPTIHERPVSFSPPPTCPPKVAISQRRKSTSFLEAQTRHFQPLLRTVGQNHLPPGSSPTNWTPEAIVMLGATANRVNRELCEMQVQPVFEPTQIYSDYRPGLVLAEEAHYFIPQETVYLAGVHYQAQVAGQYEGISYNSPVLSSPMKQISEQKPVPGGPASSSVFEFPSGQAFLVGHLQNLRLDSGPSPASPLSSISAPNSTDATHLKFHPVFVPHSAPAVLTNSNENRSNCVFEFHAQTPSSSGEGGGILPQRVYRNRQVAVDSNQEELSPQSVGLHCHLQPVTEEQRNNHAPELTISVVEPMGQIWPIGSPEYSSDSSQITSSDLSDFQSPPPTGGTAAPFGSDVSLPFIRLPQTVLQESPLFFCFPQGTTSQQVLSASYSSGGSTLHPQVIGKLSQFF

SEQ ID NO: 22 - protein, 418 aa, Mus musculus
MAHPCGGTPTYPESQIFFPTIHERPVSFSPPPTCPPKVAISQRRKSTSFLEAQTRHFQPLLRTVGQNHLPPGSSPTNWTPEAIVMLGATANRVNRELCEMQVQPVFEPTQIYSDYRPGLVLAEEAHYFIPQETVYLAGVHYQAQVAGQYEGISYNSPVLSSPMKQISEQKPVPGGPASSSVFEFPSGQAFLVGHLQNLRLDSGPSPASPLSSISAPNSTDATHLKFHPVFVPHSAPAVLTNSNENRSNCVFEFHAQTPSSSGEGGGILPQRVYRNRQVAVDSNQEELSPQSVGLHCHLQPVTEEQRNNHAPELTISVVEPMGQIWPIGSPEYSSDSSQITSSDLSDFQSPPPTGGTAAPFGSDVSLPFIRLPQTVLQESPLFFCFPQGTTSQQVLSASYSSGGSTLHPQVIGKLSQFF

SEQ ID NO: 23 - protein, 426 aa, Ratus ratus
MLIQPQSVAHPCGGTPTYPESQIFFPTIHERPVSFSPPPTCPPKVAISQRRKSTSFLEAQTRHFQPLLRTVGQNHLPPGGSPTNWTPEAIVMLGTTANRVNRELCEMQVQPVFETTQIYSDYRPGLVLAEEAHYFIPQETVYLAGVHYQAHAAGQYEGISYNSPVLSSPMKQITEQKPVPGCPASSSVFEFPSGQAFLVGHLQNLRLDSGPSPASPLSSISAPNSTDATHLKFHPVFVPHSAPAVLTHSNENRSNCVFEFHAQTPSSSSGEGGGILPQRVYRNRQVAVDSSQEELSPQSVGLHCHLQPVTEEQRNNHTPELTISVVEPMGQNWPVGSPEYSSDSSQITSSDISDFQSPPPTGGTAAPFGSDVSLPYIRLPQTVLQESPLFFCFPQGTTSQQVLSASYSSGGSALHPQVIGKLSQFF

SEQ ID NO: 24 - DNA, 24 bp, Artificial
  Forward amplification Primer
ttccagaagcattgttatttattt

SEQ ID NO: 25 - DNA, 19 bp, Artificial
  Reverse Replication Primer
cccccttgtactggcttct

SEQ ID NO: 26 - DNA, 23 bp, Artificial
  Forward Replication Primer
caccagaggccgtagttatgttg

SEQ ID NO: 27 - DNA, 24 bp, Artificial
  Reverse replication primer.
ttgaggaggcagttcttcttgatt

SEQ ID NO: 28 - DNA, 23 bp, Artificial
  Forward replication primer.
gcgcctgctgtgttaactcataa

SEQ ID NO: 29 - DNA, 24 bp, Artificial
  Reverse replication primer.
ccaaagatggggaaactctactga

SEQ ID NO: 30 - DNA, 23 bp, Artificial
  Forward replication primer.
accatcacctaaggagacagacc

SEQ ID NO: 31 - DNA, 22 bp, Artificial
  Reverse replication primer.
tgcaacaaatgtaccactctgg

SEQ ID NO: 32 - DNA, 16 bp, Artificial
  Forward replication primer.
agctcctaaatccccg

SEQ ID NO: 33 - DNA, 21 bp, Artificial
  Reverse replication primer.
gccatgtctataaataccctg

(19) United States
(12) Patent Application Publication
(10) Pub. No.: US 2006/0166207 A1
(43) Pub. Date: Jul. 27, 2006

(54) Hsan II related gene and expression products and uses thereof

(76) Inventors: Ronald G. Lafreniere, Verdun (CA); Marie-Pierre Dube, Montreal (CA); Marcia L MacDonald, Vancouver (CA); Mark E Samuels, Halifax (CA)

Correspondence Address: CARELLA, BYRNE, BAIN, GILFILLAN, CECCHI,;STEWART & OLSTEIN, 5 BECKER FARM ROAD, ROSELAND, NJ, 07068, US

(21) Appl. No.: 10/534,419

(22) PCT Filed: Nov. 12, 2003
(86) PCT No.: PCT/CA03/01716

§ 371 Date: Feb. 2, 2006

Related U.S. Application Data
(60) Provisional application No. 60/425,601, filed on Nov. 12, 2002.
(60) Provisional application No. 60/502,453, filed on Sep. 12, 2003.

(51) Int. Cl. C12Q 1/68

(52) U.S. Cl. 435/6

(57) ABSTRACT

Methods of utilizing a gene related to pain perception, herein dubbed HSN2, or its encoded protein, dubbed herein “sensorin,” for the screening and identification of agents for the treatment of pain, neuropathy and related disorders, especially small organic compounds, as well as methods of using these compounds to treat or otherwise ameliorate pain, neuropathy and related disorders in human patients. Novel polypeptides and polynucleotides, along with their nucleotide and amino acid sequences, are also disclosed.

This application claims priority of U.S. Provisional Applications 60/502,453, filed 12 Sep. 2003, and 60/425,601, filed 12 Nov. 2002, the disclosures of which are hereby incorporated by reference in their entirety.

### FIELD OF THE INVENTION

The present invention relates to methods of utilizing a gene (HSN2) involved in Hereditary Sensory and Autonomic Neuropathy, type II (HSAN II) and expression products of this gene for the screening and identification of agents, such as small organic compounds, useful in the treatment of pain, neuropathy and related disorders in human patients as well as uses thereof to treat or otherwise ameliorate such disorders.

### BACKGROUND OF THE INVENTION

The present invention relates to the medical disorder called Hereditary Sensory and Autonomic Neuropathy—Type II (“HSAN II”), which is a member of a group of hereditary pain disorders known as the Hereditary Sensory and Autonomic Neuropathies (HSAN). HSAN comprises a group of five clinically and genetically heterogeneous disorders which are mainly characterized by variable sensory and autonomic dysfunction, including absence of pain. The pathology of HSAN is characterized by degeneration of peripheral sensory neurons and it appears to be hereditary, making it of considerable scientific interest to determine which gene or genes underlies the disorder.

HSAN II is an autosomal recessive condition with distal generalized sensory loss. (See Online Mendelian Inheritance in Man (OMIM) reference *201300) Patients with HSAN II typically present with “glove and sock” distribution, meaning that they have reduced or complete loss of pain, temperature and touch sensations in the lower legs and feet as well as hands and forearms. Age of onset is quite early, usually in the first decade of life. They first complain of a numbness in their extremities which is aggravated by the cold, after which pain sensation is reduced. Patients have difficulty with handling small objects (like coins) in their pockets due to the lack of sensation. Some families appear to have a slowly progressive form, where the lack of sensation slowly grows up the legs and arms until in some cases, the patients have reduced sensations in their trunkal regions while others do not Reduced sensation is confined just to the lower legs (usually below the knees), forearms and hands. Muscle atrophy (particularly in calves, and in between the thumb and first finger), ulcerations and infections are major secondary features. Amputations of fingers, toes and sometimes feet and lower limbs, resulting from infections, are not uncommon. Reflexes are diminished or lost. There is very little, if any, autonomic involvement, so sweating and blood pressure are normal.

The pathology of HSAN II appears to be tied to peripheral nerve degeneration. Biopsy reveals that within the sural nerve there is a severe loss of myelinated axons, and a lesser loss of nonmyelinated fibres. No cutaneous sensory receptors or nerve fibers are seen. (Axelrod, F. B. “Autonomic and sensory disorders”. Chapter 117; pages 3146-3161 in Emery and Rimoin's Principles and Practice of Medical Genetics (Fourth Edition) Edited by: David L. Rimoin, J. Michael Connor, Reed E. Pyeritz, Bruce R. Korf. Published: Harcourt Publishers Limited London 2002).

The genetic basis of HSAN II is yet to be determined. No genetic loci have been confirmed, and no mutations in suggested candidate genes have been identified.

The HSAN II gene (dubbed herein “HSN2”) would present a novel target for therapeutic agents, The functional validation disclosed herein shows that the activity of the gene/protein is involved in very specific human disease processes. Therapeutic agents which modulate the biological activity of the HSAN II gene or its corresponding protein are novel therapeutic agents for the treatment of pain, neuropathy and related disorders.

The screening assays disclosed herein select from a large library of compounds those compounds that interact with, bind to or otherwise modulate the activity of the target gene/protein. A wide body of commercial literature describes the screening of chemical libraries of diverse potential therapeutic agents to identify such potential therapeutic agents.

Therapeutic agents for treatment of pain fall into two main classes—the NSAIDs (non-steroidal anti-inflammatory drugs) and the opioids. NSAIDs treat pain in a way similar to the mechanism of aspirin, the most well-known and oldest member of the class. Common NSAIDs include acetaminophen, ibuprofen and naproxen. These drugs mainly inhibit the body's ability to synthesize prostaglandins. The common mechanism of action for all NSAIDs is the inhibition of the enzyme cyclooxgenase (COX). A major commercial success has been achieved with specific inhibitors of COX-2, such as Celebrex™ from Pharmacia/Pfizer, and Vioxx™ from Merck & Co.

Opioids act through the opioid receptor family. These drugs include the weak opioids such as codeine and Tylenol 3, and strong opioids such as morphine and methadone. Some are long acting, others are of short duration. Opioid analgesics have a tendency to addiction and dependency, and so are not preferred for long-term or chronic pain management

Outside of the NSAIDs and opioids, there are a number of other suggested analgesic agents in clinical trials (i.e. not yet approved for marketing) which are believed to have alternative targets. Some clinical trials are attempting to establish that central neuropathic pain may respond to ion channel blockers such as blockers of calcium, sodium and/or NMDA (N-methyl-D-aspartate) channels. The literature provides substantial pre-clinical electrophysiological evidence in support of the use of NMDA antagonists in the treatment of neuropathic pain. Such agents also may find use in the control of pain after tolerance to opioid analgesia occurs, particularly in cancer patients.

Current treatment of neuropathies first entails identifying the cause, then treating the cause if possible. There is a substantial list of disorders that can result in neuropathies, including diabetes and other diseases. Most neuropathies (except the genetic forms) are treatable by treating the root causes, though most treatments are only partially effective. For example vitamin deficiency is treated with vitamins; Guillain-Barre and CIDP are treated with gamma globulins and plasmapheresis. For genetic forms of neuropathy, current clinical practice is to manage patients by supporting their efforts to avoid self-inflicted injury.

In accordance with the present invention, the identification of the hereditary basis for HSAN II facilitates developing more potent agents for treating pain, neuropathy, and related disorders. The underlying genetic mutation provides a therapeutic target for novel therapeutic agents. This therapeutic target permits identification and discovery of more effective analgesics and other therapeutic agents, as well as new methods and compositions for diagnosis of HSAN II distinguish between types of inherited pain disorders.

### BRIEF SUMMARY OF THE INVENTION

In one aspect, the invention relates to the nucleic acid sequence for HSN2, including the genomic sequence, mRNA or cDNA, polymorphic, allelic, isoforms (adult, neo-natal, etc.) and mutant forms thereof, and nucleic acid constructs of the gene, including vectors, plasmids and recombinant cells and transgenic organisms containing or corresponding to HSN2 (or knock-outs thereof.

In another aspect, the invention relates to the gene product of HSN2 (sensorin), including the polypeptide, protein, and amino acid sequence, and the polymorphic, allelic, isoforms (adult, neo-natal, etc.) and mutant forms thereof, and recombinant cells and transgenic organisms wherein this polypeptide or a polypeptide corresponding thereto is expressed.

In another aspect of the present invention, the HSN2 gene or protein is incorporated into a screening assay whereby test compounds (potential therapeutic agents) are evaluated for their ability to modulate HSN2 gene expression or sensorin activity, thereby identifying modulators of the gene or protein and thus potential therapeutic agents.

In preferred embodiments, the polynucleotide whose expression is to be measured or monitored is present in an intact cell, preferably a mammalian cell, most preferably a peripheral neuron, and may include a recombinant cell. Such polynucleotide may also be present outside of a cell and the expression may be measured in vitro.

In another aspect, the present invention relates to a method for identifying an agent that modulates the activity of a polypeptide encoded by a polynucleotide as disclosed herein.

In a further aspect, the present invention relates to a method for identifying an agent for the treatment of pain, neuropathy or a related disorder in an animal. Preferably, the animal is a mammal, such as a human being. In specific embodiments, the pain stimulus is a heat stimulus and reaction or sensitivity to hot and/or cold may be measured. In another embodiment, an electrical stimulus may be used.

In a preferred embodiment of the invention; the compound identified which modulates HSN2 (or sensorin) is selective for the target ahead of related genes or proteins.

In a further aspect, the present invention relates to a method for treating a condition in an animal afflicted with chronic pain, neuropathy or a related disorder comprising administering to said animal an effective amount of an agent first identified by an assay method of the invention. Preferably, said animal is a human patient. Such determined agents may also be applied to alternative or additional indications beyond pain, neuropathy or other disorders, which are found to be treatable by modulating HSN2 or sensorin activity.

In a further aspect, the present invention relates to compounds that modulate HSN2 or sensorin. These compounds include antibodies, antisense compounds, gene therapy vectors and proteins, and small molecule organic compounds. The invention also comprises the use of these compounds in the treatment of pain, neuropathy and related disorders.

### BRIEF DESCRIPTION OF THE DRAWINGS

FIG. 1a shows the pedigree for a family designated HSAN4 showing family relationships and haplotypes. FIG. 1b shows the pedigree for a family designated HSAN3 showing family relationships and haplotypes. Squares and circles represent males and females, respectively. Filled symbols indicate individuals with HSAN II. Symbols with an “N” indicate individuals diagnosed as normal, and clear symbols and symbols with a question mark indicate individuals who have not been diagnosed

FIG. 2. Domain prediction of the HSN2 protein, sensorin. TM topology was predicted using TMPred.

FIG. 3. Peptide sequence alignment of human, mouse, rat, fugu and tetraodon sensorin proteins.

FIG. 4. Peptide sequence alignment of human and Fugu sensorin.

FIG. 5. Peptide sequence alignment of Fugu and Zebrafish orthologs of sensorin.

FIGS. 6A-6E shows the sequences of HSN2 from various sources. In all cases, cDNA sequences are shown in capital letters. Start and stop codons are underlined.

FIG. 6A shows a sequence (SEQ ID NO: 1) for wild-type human HSN2 putative cDNA (with ends based on human ESTs).

FIGS. 6B-1 and 6B-2 shows the promoter sequence (SEQ ID NO: 6) for human HSN2 (presumably there only 1 promoter used and it is not associated with a CpG-rich island; this corresponds to the segment of PRKWNK1 intron 8 upstream of the HSN2 start codon; the transcription start site and cDNA sequence (shown in capital letters) for HSN2 is tentative and based on 1 EST sequence; the translation start site, and start ATG, is the last 3 bases.

FIG. 6C shows the nucleotide sequence (SEQ ID NO: 7) of wild-type mouse HSN2 cDNA predicted (5′ and 3′ ends not determined). The putative polypeptide is SEQ ID NO: 8.

FIG. 6D shows the nucleotide sequence (SEQ ID NO: 9) for wild-type rat HSN2 (with the genomic sequence defined by 5′ and 3′ ends in EST BF522762/AI578184, and containing a polyA tail).

FIG. 6E-1 to 6E-6 show the genomic structure and sequence of the human HSN2. (SEQ ID NO. 12), with the figures following in sequence. The HSN2 gene is a single unspliced exon found in intron 8 of the PRKWNK1 gene (also called XH03, below). Here, italics=interspersed repeats, uppercase=exons, bold=exons (or part of exons) found in common isoforms and underlined=start and stop codons, or upstream in-frame stop codon.

FIGS. 7A-7C show the sequences of cDNAs for three separate mutations identified in individuals afflicted with HSAN II. Start and stop codons are underlined. FIG. 7A shows mutation 1 (SEQ ID NO: 14) as human cDNA with mutation c.594delA. FIG. 7B shows mutation 2 (SEQ ID NO: 15) as human cDNA with mutation c.918insA. FIG. 7C shows mutation 3 (SEQ ID NO: 16) as human cDNA with mutation c.943C>T.

FIG. 8 shows three in-frame candidate translation initiation codons.

FIG. 9 shows a sequence alignment between the human and pig peptide sequences.

### DEFINITIONS

The following terms have their indicated meaning unless expressly stated otherwise herein.

As used herein, the term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:
Percent Identity=100[1−(C/R)]
wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each: gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the hereinabove calculated Percent Identity is less than the specified Percent Identity.

As used herein, the terms “portion,” “segment,” and “fragment,” when used in relation to polypeptides, refer to a continuous sequence of nucleotide residues, sequence forms a subset of a larger sequence. Such terms include the products produced by treatment of said polynucleotides with any of the common endonucleases, or any stretch of polynucleotides that could be synthetically synthesized. These may include exonic and intronic sequences of the corresponding genes.

As used herein the term “encode” and its derivatives mean “can be transcribed into” or “can be translated into” so that a gene or other deoxy polynucleotide sequence can be described in terms of its ability to encode an RNA or polypeptide while the latter terms relate to materials that can be described in terms of their being encoded by said gene or deoxy polynucleotide. In the same way, an RNA can be said to encode a polypeptide while said polypeptide can be described as being encoded by said RNA.

The words “therapeutic target” mean a gene, an RNA or polypeptide for which therapeutic intervention is achieved with agents that modulate the activity of said gene, RNA or protein. “Modulate” means to increase, to decrease, or to otherwise change the expression or activity, especially of a therapeutic target.

“HSN2 activity” or “sensorin activity” as used herein, especially relating to screening assays, is to be interpreted broadly and contemplates all directly or indirectly measurable and identifiable biological activities of the HSN2 gene, gene products and sensorin. The HSN2 gene is thus involved with functions related to Hereditary Sensory and Autonomic Neuropathy—Type II (“HSAN II”) and is, in the past, has also been called the “HSAN II Susceptibility Gene.”

The term “polynucleotide” is used interchangeably with “gene”, “cDNA”, “mRNA”, “oligonucleotide”, and “nucleic acid”.

The terms “polypeptide,” “peptide,” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers.

The term “agent” is used interchangeably with the term “compound” and likewise the term “test agent” is used interchangeably with the term “test compound”.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “mammal” refers to a member of the zoological class Mammalia. Examples of mammals include, without limitation, humans, primates, chimpanzees, mice, rats, rabbits, sheep, and cows.

The term “treat” or “treatment” encompasses therapeutic treatment, preventive treatment and protective treatment. Such protective treatment includes treatment of patients, especially to achieve analgesia.

As used herein, the term “correspond” applied to a gene or polynucleotide, such as a DNA, means that the gene has the indicated nucleotide sequence or that it encodes substantially the same RNA as would be encoded by the indicated sequence, the term “substantially” meaning at least 95% identical as defined elsewhere herein.

The term “DNA segment” refers to a DNA polymer, in the form of a separate fragment or as a component of a larger DNA construct, which has been derived from DNA isolated at least once in substantially pure form, i.e., free of contaminating endogenous materials and in a quantity or concentration enabling identification, manipulation, and recovery of the segment and its component nucleotide sequences by standard biochemical methods, for example, using a cloning vector. Such segments are provided in the form of an open reading frame uninterrupted by internal nontranslated sequences, or introns, which are typically present in eukaryotic genes. Sequences of non-translated DNA may be present downstream from the open reading frame, where the same do not interfere with manipulation or expression of the coding regions.

“Isolated” in the context of the present invention with respect to polypeptides (or polynucleotides) means that the material is removed from its original environment (e.g., the natural environment if it is naturally occurring). For example, a naturally-occurring polynucleotide or polypeptide present in a living organism is not isolated, but the same polynucleotide or polypeptide, separated from some or all of the co-existing materials in the natural system, is isolated. Such polynucleotides could be part of a vector and/or such polynucleotides or polypeptides could be part of a composition, and still be isolated in that such vector or composition is not part of its natural environment. The polypeptides and polynucleotides of the present invention are preferably provided in an isolated form, and preferably are purified to homogeneity.

The polynucleotides, and recombinant or immunogenic polypeptides, disclosed in accordance with the present invention may also be in “purified” form. The term “purified” does not require absolute purity; rather, it is intended as a relative definition, and can include preparations that are highly purified or preparations that are only partially purified, as those terms are understood by those of skill in the relevant art. For example, individual clones isolated from a cDNA library have been conventionally purified to electrophoretic homogeneity. Purification of starting material or natural material to at least one order of magnitude, preferably two or three orders, and more preferably four or five orders of magnitude is expressly contemplated. Furthermore, claimed polypeptide which has a purity of preferably 0.001%, or at least 0.01% or 0.1%; and even desirably 1% by weight or greater is expressly contemplated.

The term “coding region” refers to that portion of a gene which either naturally or normally codes for the expression product of that gene in its natural genomic environment, i.e., the region coding in vivo for the native expression product of the gene. The coding region can be from a normal, mutated or altered gene, or can even be from a DNA sequence, or gene, wholly synthesized in the laboratory using methods well known to those of skill in the art of DNA synthesis.

In accordance with the present invention, the term “nucleotide sequence” refers to a heteropolymer of deoxyribonucleotides. Generally, DNA segments encoding the proteins provided by this invention are assembled from cDNA fragments and short oligonucleotide linkers, or from a series of oligonucleotides, to provide a synthetic gene which is capable of being expressed in a recombinant transcriptional unit comprising regulatory elements derived from a microbial or viral operon.

The term “expression product” means that polypeptide or protein that is the natural translation product of the gene and any nucleic acid sequence coding equivalents resulting from genetic code degeneracy and thus coding for the same amino acid(s).

The term “fragment,” when referring to a coding sequence, means a portion of DNA comprising less than the complete coding region whose expression product retains essentially the same biological function or activity as the expression product of the complete coding region.

The term “primer” means a short nucleic acid sequence that is paired with one strand of DNA and provides a free 3′OH end at which a DNA polymerase starts synthesis of a deoxyribonucleotide chain.

The term “promoter” means a region of DNA involved in binding of RNA polymerase to initiate transcription.

The term “open reading frame (ORF)” means a series of triplets coding for amino acids without any termination codons and is a sequence (potentially) translatable into protein.

As used herein, reference to a DNA sequence includes both single stranded and double stranded DNA. Thus, the specific sequence, unless the context indicates otherwise, refers to the single strand DNA of such sequence, the duplex of such sequence with its complement (double stranded DNA) and the complement of such sequence.

### DETAILED DESCRIPTION OF THE INVENTION

In accordance with the present invention, the human HSN2 Gene, is disclosed. This gene, when mutated, results in Hereditary Sensory and Autonomic Neuropathy—Type II (“HSAN II”). This gene is designated herein as HSN2 (but in the past has sometimes been referred to as SENS or XH18 are other designations). The polypeptide is referred to as HSN2 Protein or HSN2 peptide. In the past, it has also been referred to as HSAN II polypeptide, HSN2 polypeptide, HSANII peptide. It is also referred to as sensorin, although this latter term has been applied to a minor unrelated protein in the past. This naming is not essential to use of the invention claimed herein.

Thus, the present invention provides a gene and its corresponding protein related to insensitivity to pain, neuropathy and related disorders, referred to as HSN2 and its corresponding polypeptide sensorin or HSN2 protein.

The wild type nucleic acid sequence of human HSN2 is provided at SEQ ID NO. 1. The wild type amino acid sequence of human HSN2 protein is set forth in SEQ ID NO: 2.

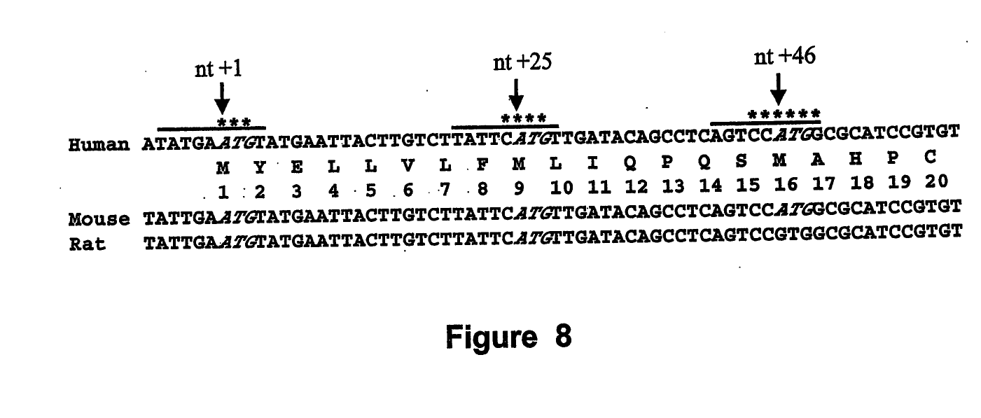

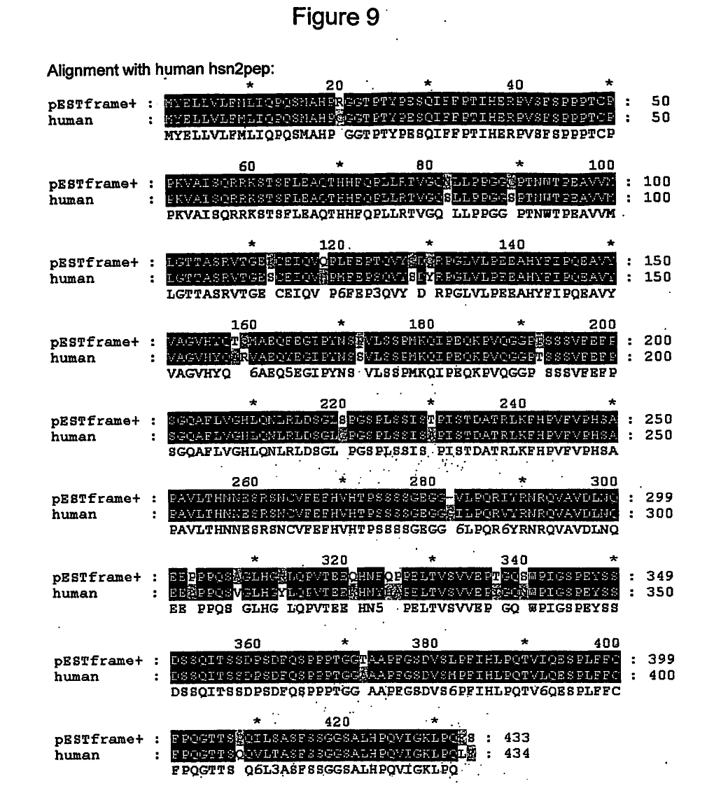

Three in-frame candidate translation initiation codons have been identified (FIG. 8). The 3^{rd} ATG is closest to the optimal Kozak consensus C-C-[AG]-C-C-A-T-G-G context for initiation of translation, but is not conserved in the rat sequence. Those skilled in the art are capable of characterizing the most efficient translation initiation context by generating three HSN2 cDNA expression vectors, each containing different starting site combinations including one, two or three ATG codons.

The human HSN2 gene maps to the HSAN II candidate interval at 12p13.33. The gene encodes a full-length conserved peptide of 434 amino acids. It comprises a single unspliced axon which maps to intron 8 of the PRKWNK1 gene and is transcribed from the same strand as PRKWNK1. HSN2-encoded protein may be the full length PRKWNK1 protein with a special exon. Thus, the present invention can also encompass a full length PRKWNK1 protein (including cDNA) containing the HSN2 exon.

In one aspect, the present invention relates to an isolated polynucleotide comprising a polynucleotide having a nucleotide sequence with at least 60% identity to, preferably at least 70% identity to, more preferably at least 78% identity to, even more preferably at least 90% identity to, most preferably at least 95% identity to, and especially at least 98% identity to a sequence selected from the group consisting of SEQ ID NO: 1, 6, 7, 9 and 12 wherein said isolated polynucleotide encodes a polypeptide that binds to an antibody specific for a polypeptide having the amino acid sequence of SEQ ID NO: 2. In an especially preferred embodiment, the isolated polynucleotide comprises the nucleotide sequence of SEQ ID NO: 1, 6, 7, 9 or 12.

In another aspect, the present invention relates to an isolated polypeptide comprising a polypeptide having an amino acid sequence with at least 90% identity to, preferably at least 95% identity to, more preferably at least 98% identity to and most preferably having the amino acid sequence selected from the group consisting of SEQ ID NO: 2, 8, 10, 11 and 13 wherein said polypeptide binds to an antibody specific for a polypeptide having the amino acid sequence of SEQ ID NO: 2.

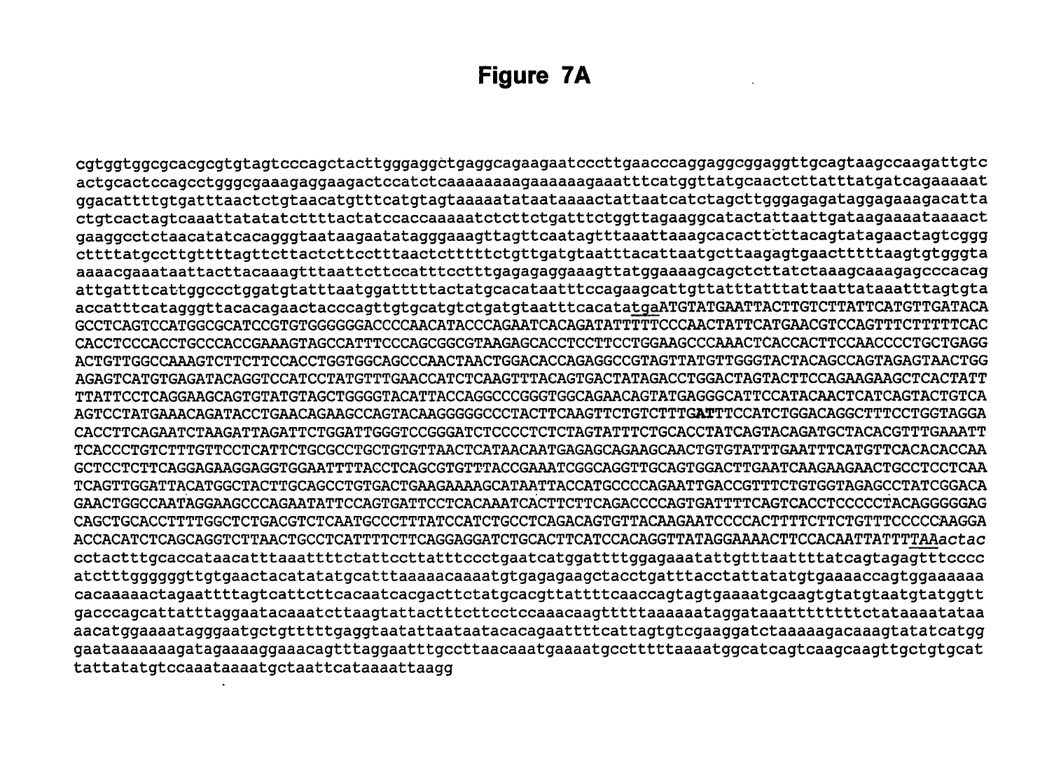

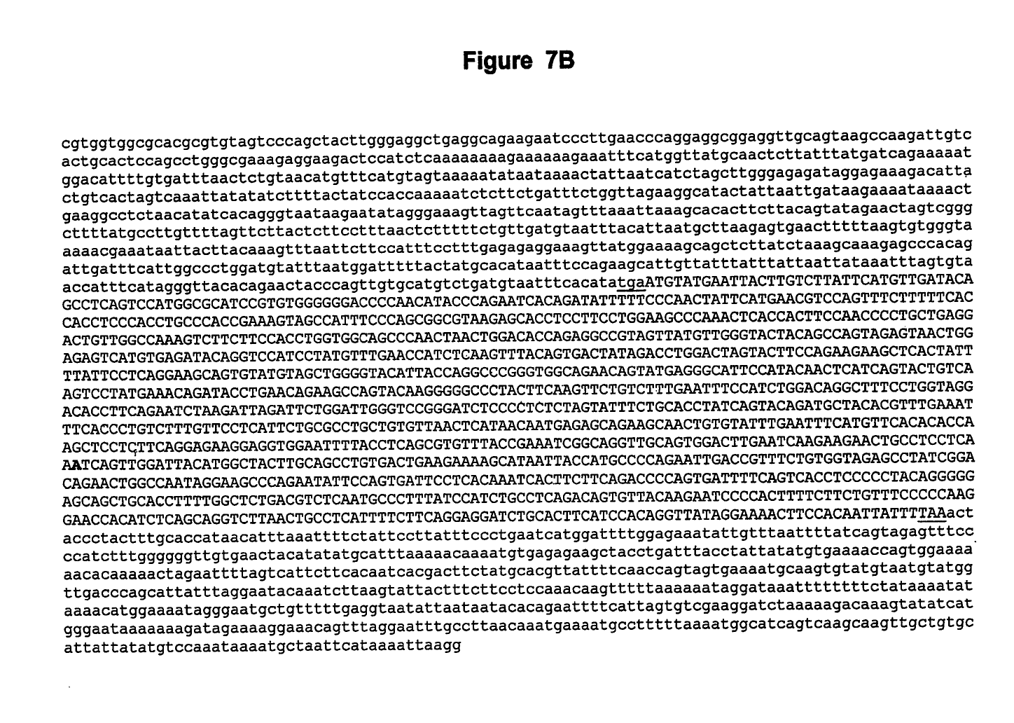

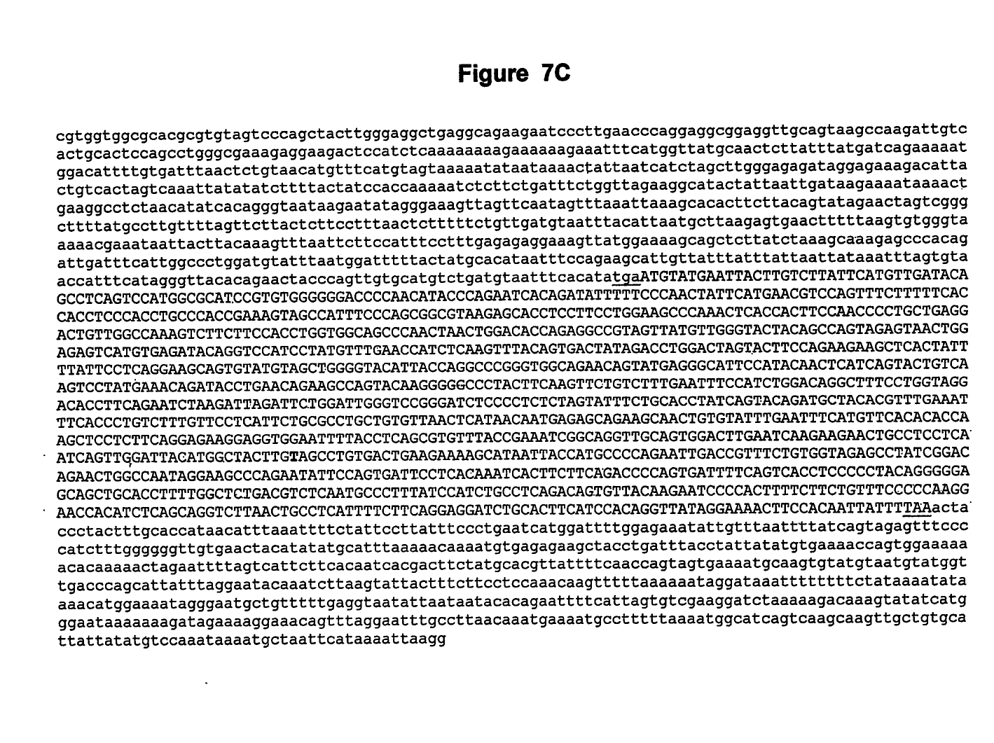

As disclosed herein, three separate mutations were identified in individuals affected with HSAN II, none of which are found in the homozygous state in unaffected individuals (see Table 3, SEQ ID NO: 3 to 5). Each mutation affects the open reading frame (ORF) of the HSN2 gene. The cDNA sequences for these mutations are shown in FIG. 7 and may be summarized as follows:

Mutation 1. c.594delA; frameshift in codon 198, protein truncated to 206 aa: A deletion of an A (adenine) at position 594 in the nucleotide sequence (relative to the start ATG), found homozygous in the HSAN4-70 sample, causes a frameshift in codon 198 leading to premature truncation of a 206 aa peptide. The predicted amino acid sequence of the truncated human sensorin sequence from Mutation 1 is set forth at SEQ ID NO. 3. The same mutation was also later found to be responsible for the disease in the HSAN3 family. This deletion segregated with disease in other members of the HSAN3 and HSAN4 families. The mutation was not detected in any of 217 normal chromosomes from individuals of European descent.

Two different mutations were found in the HSAN5-302 sample.

Mutation 2. c.918insA, frameshift in codon 307, protein truncated to 318 aa. This mutation is a 1 bp insertion of an A between bases 918-919, causing a frameshift in codon 307, and leading to premature truncation to produce a 318 aa (amino acid) peptide. The predicted amino acid sequence of the truncated human sensorin sequence from Mutation 2 is set forth at SEQ ID NO.4

Mutation 3. c.943C>T; Gln315stop; protein truncated to 314 aa. This mutation is a C>T change at position 943 that changes codon 315 (CAG, encoding glutamine) to a TAG stop codon, therefore prematurely truncating the protein at 314 aa. Each of these mutations was not found in the control sample, dbSNP database, nor available genomic and EST sequences. The predicted amino acid sequence of the truncated human sensorin sequence from Mutation 3 is set forth at SEQ ID NO. 5. The same mutation was also later found to be responsible for the disease in the HSAN6 family.

Segregation of mutations in HSN2 with affected HSAN II patients has now been confirmed in 5/5 affecteds and 0 control (unaffected) individuals.

Promoter: A sequence containing the promoter sequence is set forth at SEQ ID NO. 6. It includes the portion of PRKWNK1 intron 8 upstream of the HSN2 translation initiation site. There are a number of DNA elements that are conserved between human and mouse in the region upstream of the presumed transcription initiation site that may act as promoter elements, including silencers and enhancers, and other transcription factor binding sites.

Other single nucleotide polymorphisms (SNPs) and variations: there is only 1 documented SNP (rs1054186) in the HSN2 gene, which corresponds to a C>T change at position 1690 of the cDNA, located in the 3′UTR. This putative SNP may not be real, as it is found in only one published expressed sequence tag (EST)and the quality of the sequencing is too poor to evaluate additional SNPs in this gene.

BAC contig: The HSN2 gene maps to BAC (Bacterial Artificial Chromosome) clone AC004765, and there are no gaps in the genomic structure. Based on the structure of the HSN2 gene determined above, the gene spans a maximum of 3 kb, consists of a single exon mapping within intron 08 of the XH03 gene (PRKWNK1), and is transcribed from the same strand as XH03, namely tel 5′-HSN2-3′ cen in 12p13.33. There are probably no additional spliced exons, since these would interfere with splicing of XH03. The position of HSN2 within an intron of the PRKWNK1 gene is conserved in human, mouse, rat and fish (form example, Fugu and Tetraodon).

Markers: D12S91 is the closest marker, mapping ˜29 kb telomeric to HSN2.

Genomic sequence: the full human genomic sequence illustrating the nucleic acid sequence around HSN2 is set forth at SEQ ID NO. 12.

Conservation of HSN2 in Other Organisms.

Orthologs of the human HSN2 gene were found in mouse, rat and fish (Takifugu rubripes (“Fugu”), Danio rerio (“Zebrafish”), and Tetraodon nigroviridis (“Tetraodon”).)

A conserved mouse ORF, and flanking sequences were identified from a mouse BAC AC113092. The nucleic acid sequence of mouse HSN2 is set forth at SEQ ID NO. 7. The translated amino acid sequence of mouse HSN2 is set forth at SEQ ID NO. 8. No mouse ESTs of HSN2 were identified in a comprehensive public database search.

Likewise the rat ortholog was identified from rat genomic sequences, and verified with the sequence of one rat EST (BF522762). This EST was used to identify the conserved rat ORF based on the genomic sequence found in BAC sequences AC106348 and AC106932. The nucleic acid sequence of wild-type rat HSN2 is set forth at SEQ ID NO. 9. The translated amino acid sequence of wild-type rat sensorin is set forth at SEQ ID NO. 10.

Analysis of human, mouse and rat orthologs. The human protein is 87% identical to mouse, and 85% identical to rat. The upstream stop codon immediately preceding the start ATG is conserved in human, mouse and rat.

Fugu (Takifugu rubripes): The human sensorin peptide sequence was used to screen the fugu genome by sequence similarity searching. One hit gave an E value score of 1e-25 against a Fugu genomic fragment (CAAB01000768). The sequence was extracted and the conserved ORF is set out at SEQ ID NO. 11.

Zebrafish (Danio rerio): The Fugu peptide sequence was used to screen the non-human non-mouse EST database using tBLASTn to identify additional orthologs. An EST from a zebrafish adult retina cDNA library was found (BG304539) which gave a partial protein sequence which is well-conserved to sensorin in Fugu. There were no additional zebrafish ESTs in GenBank.

Tetraodon (Tetraodon nigroviridis): The Fugu peptide sequence was used to screen the publicly available Tetraodon genomic scaffolds (http://fugu.hgmp.mrc.ac.uk/blast/) and a partial sequence of the Tetraodon sensorin ortholog was identified. (SEQ ID No. 13)

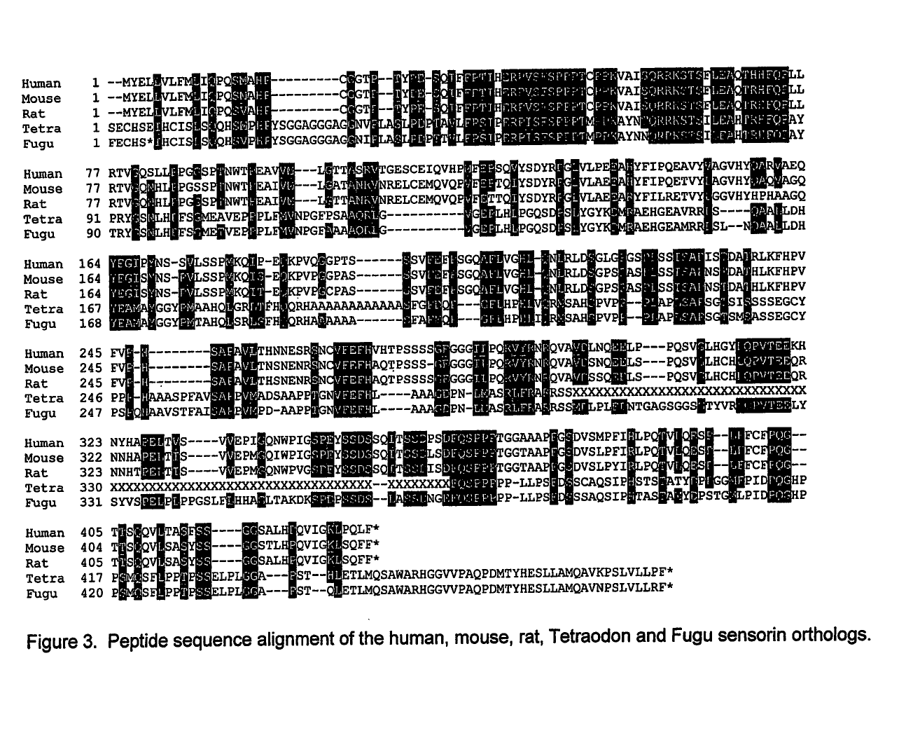

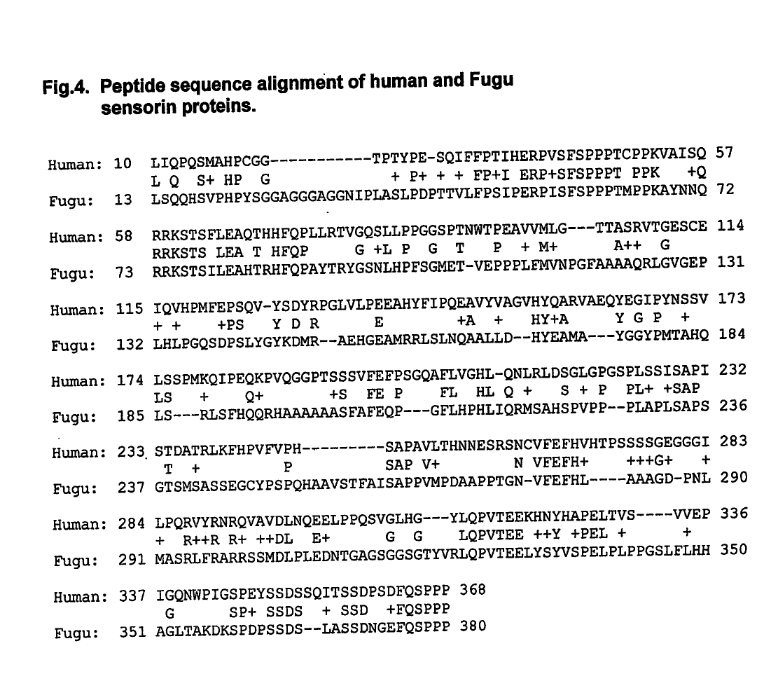

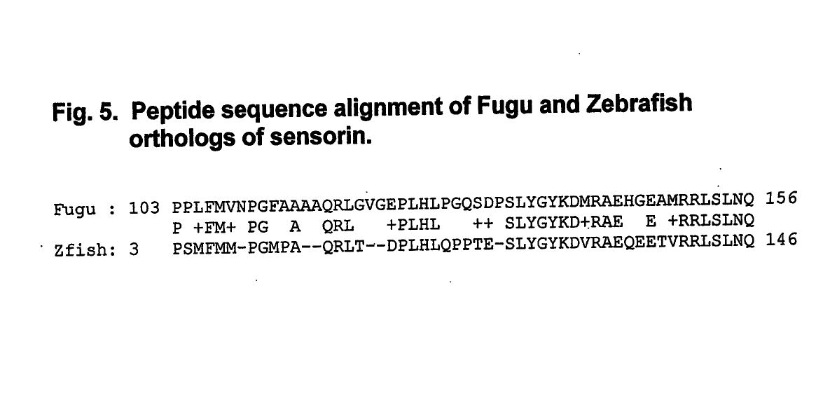

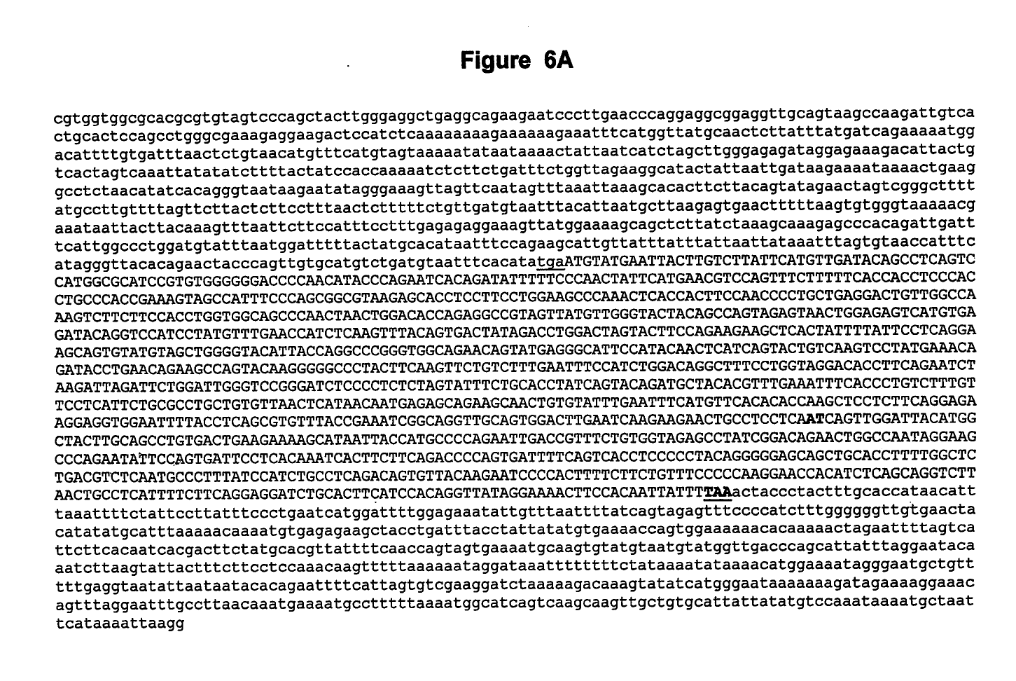

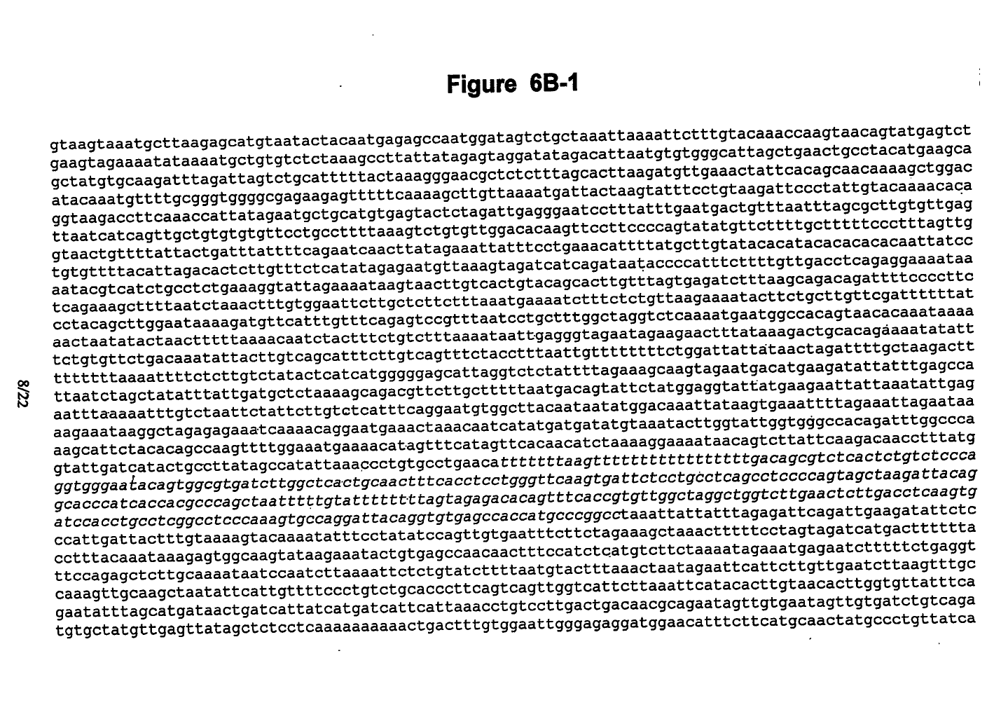

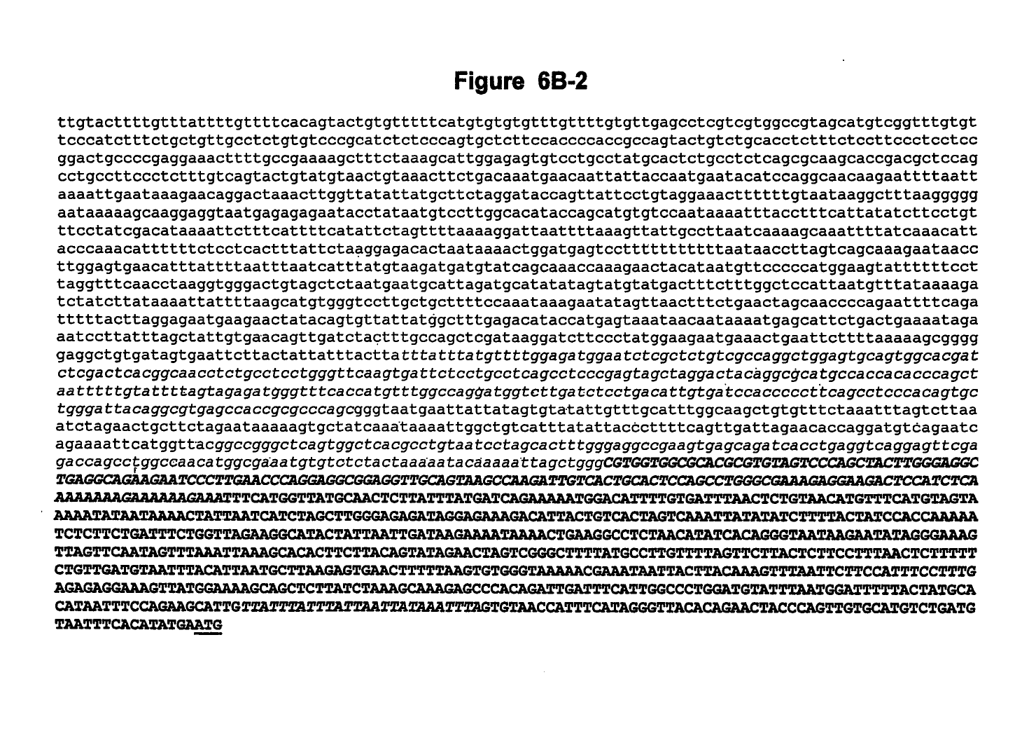

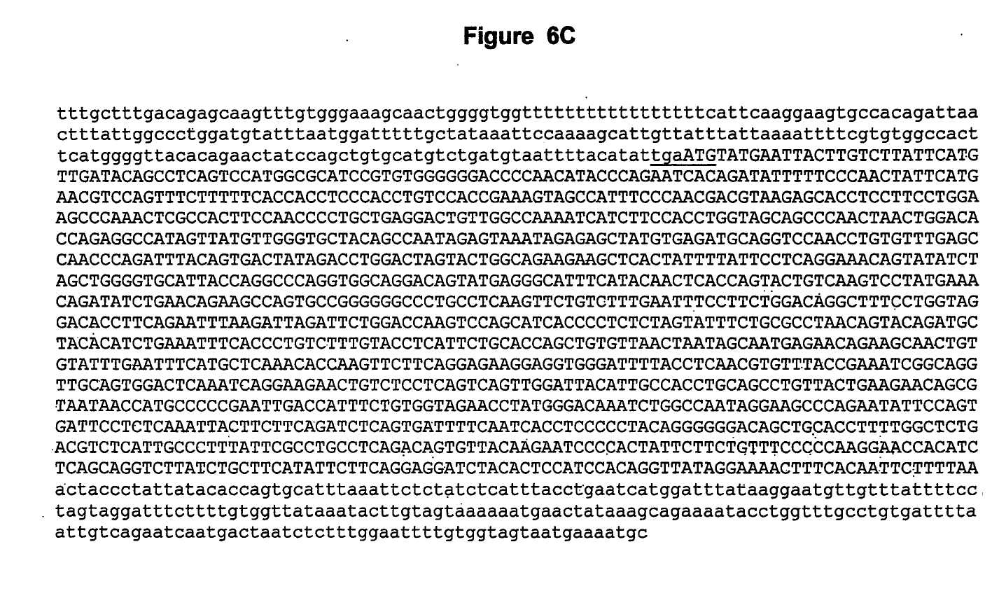

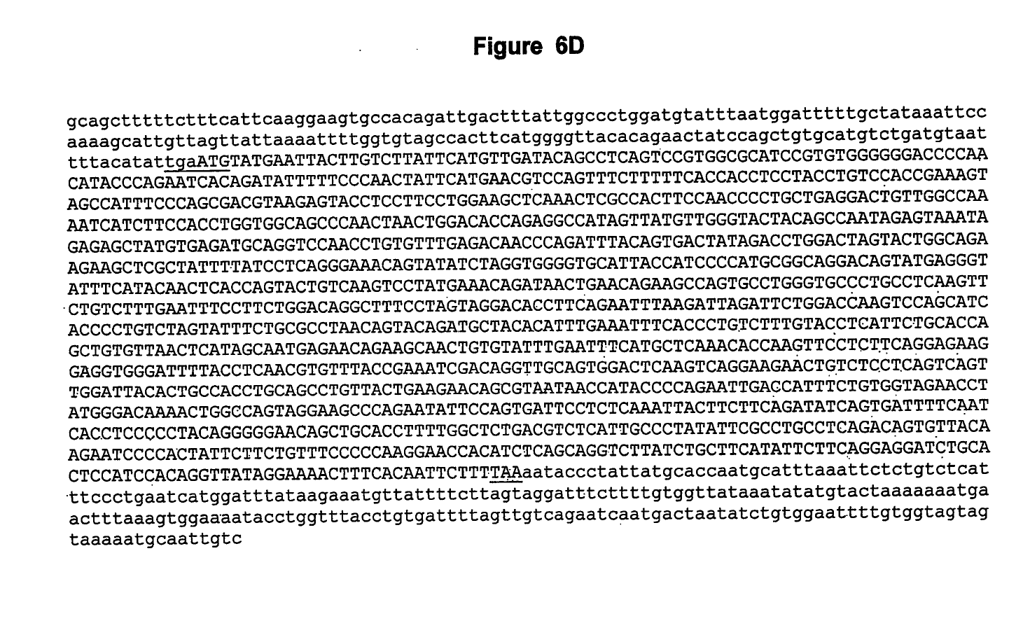

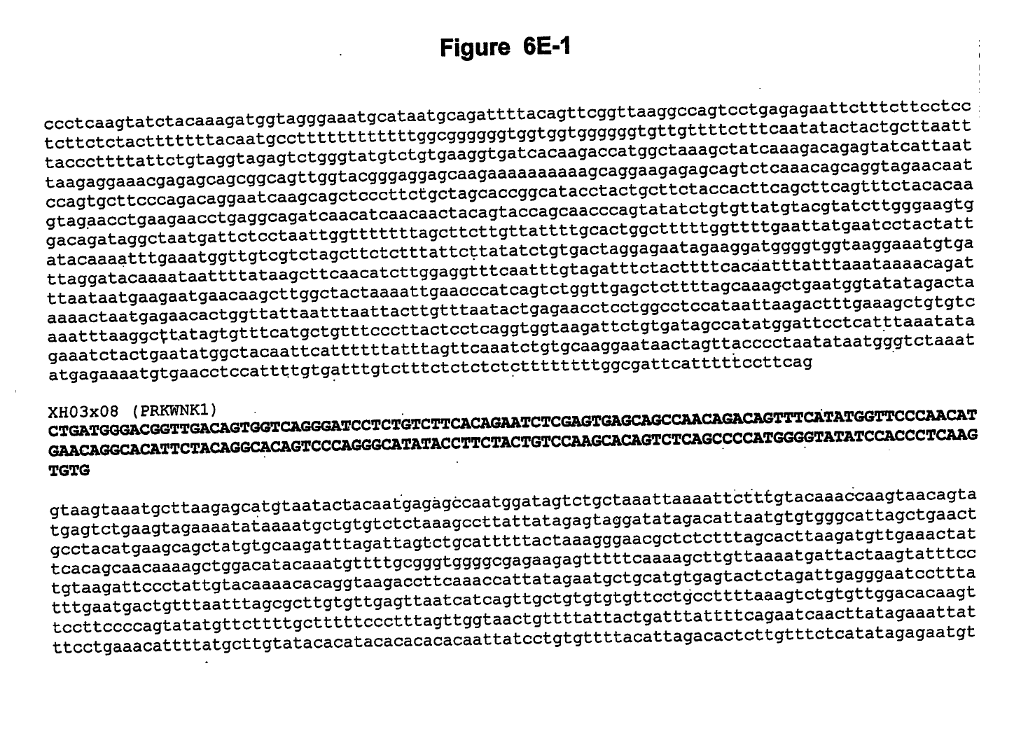

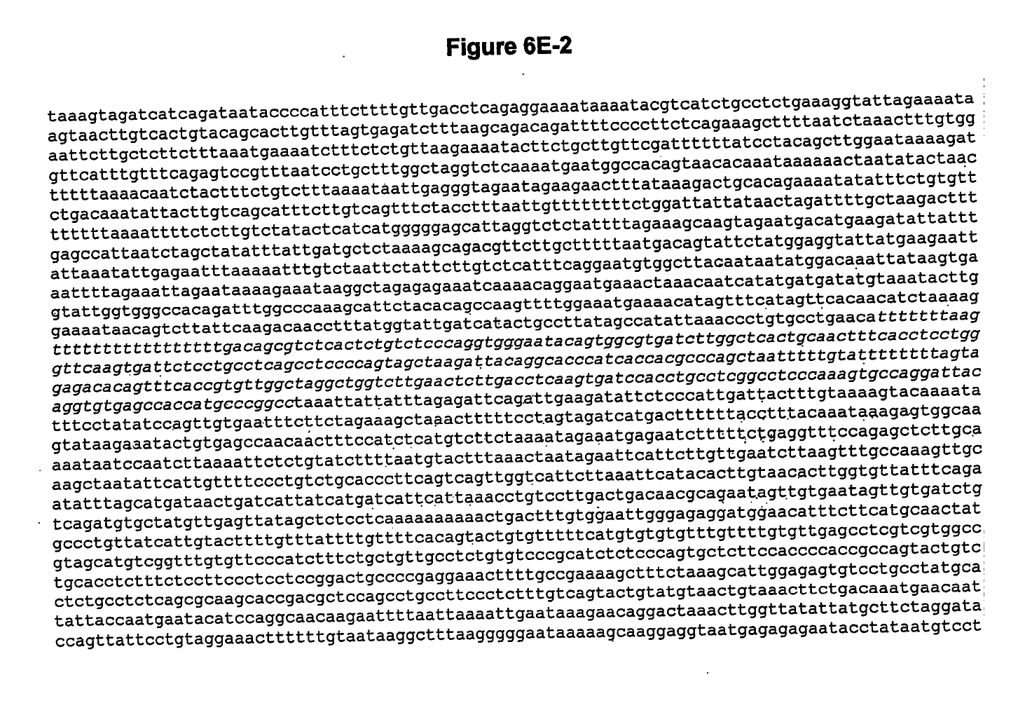

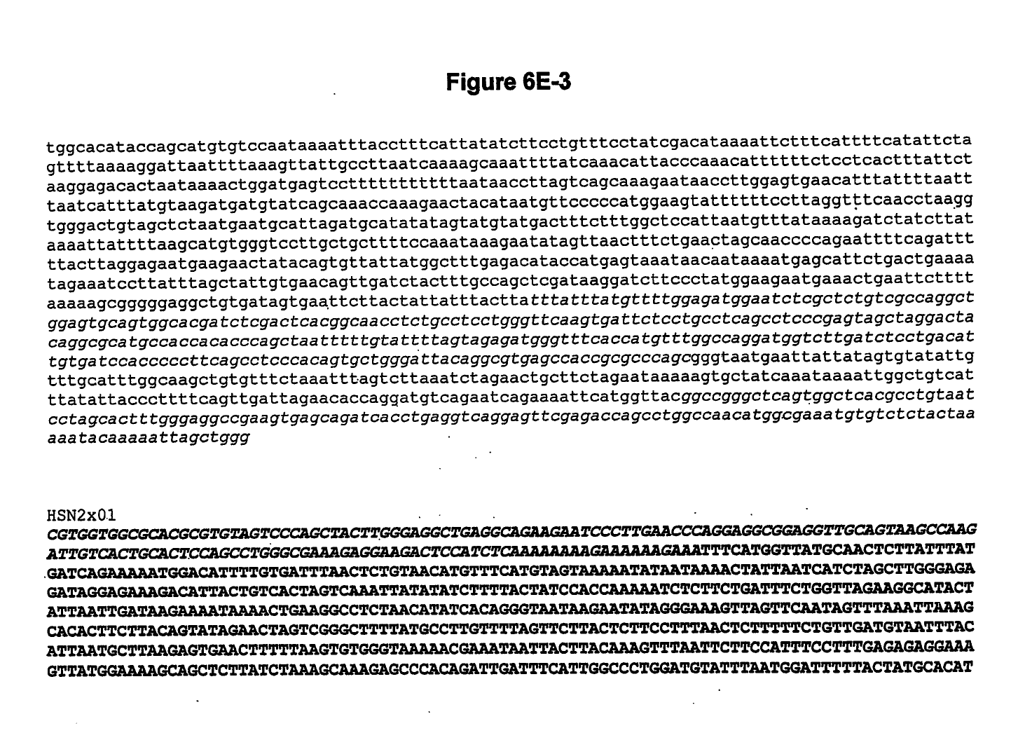

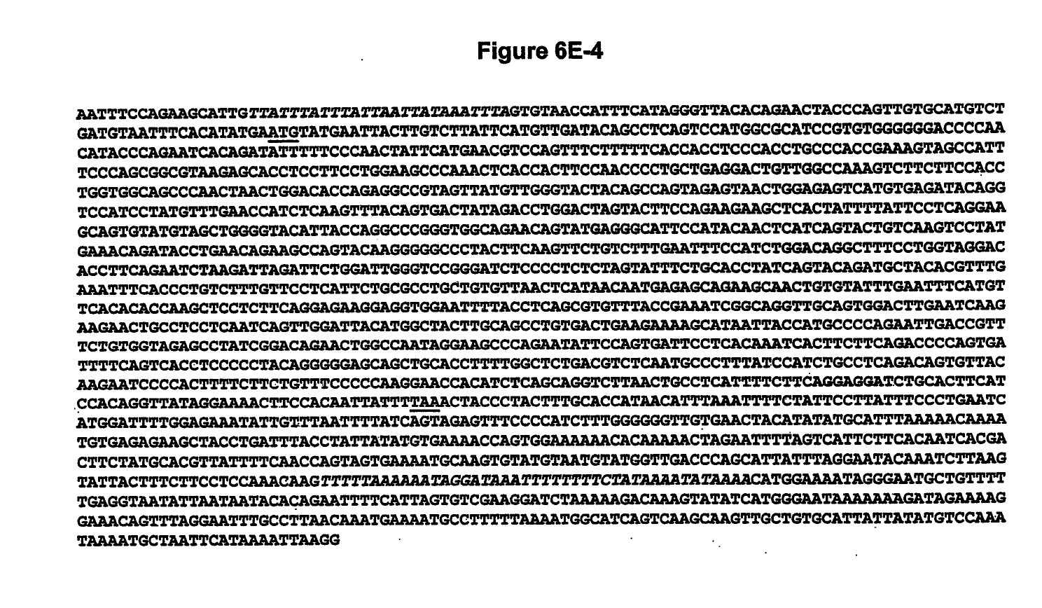

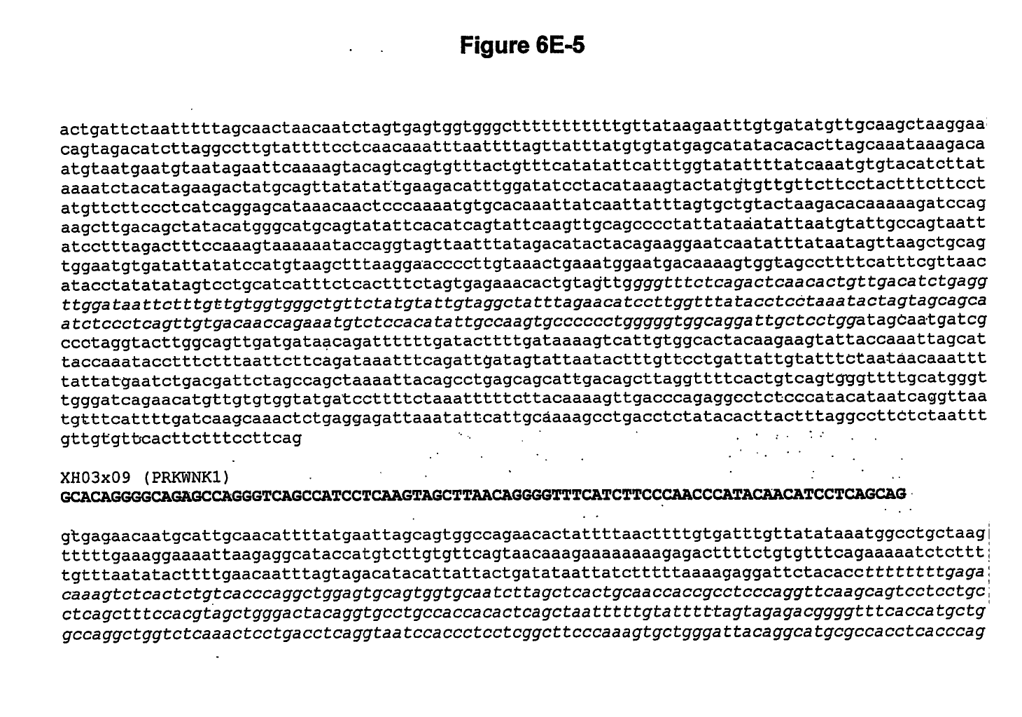

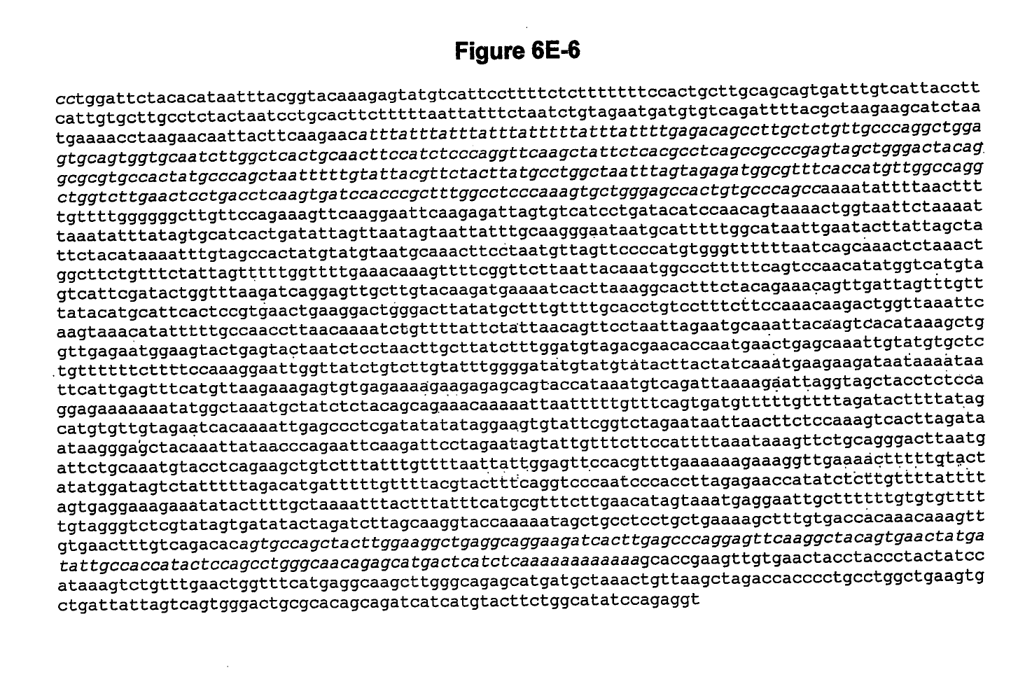

Alignment of the human, mouse, rat, Fugu, Tetraodon and zebrafish sensorin amino acid sequences is shown in FIG. 3.

Evaluation of Sensorin Function

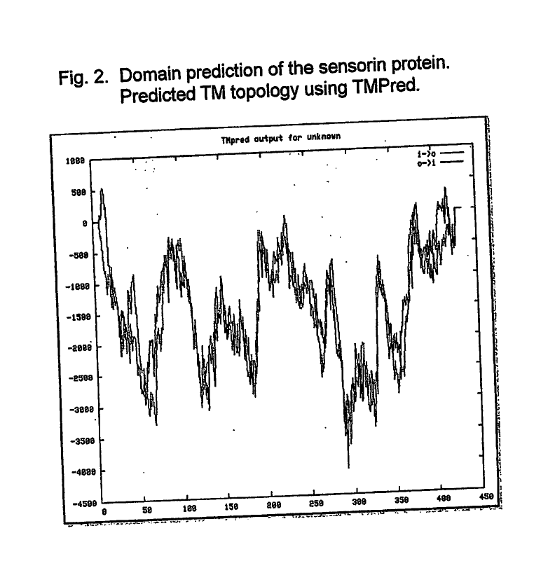

The membrane topology of the sensorin was assessed using TMPred (located at www.ch.embnet.org/software/TMPRED_form.html), which predicted a single N-terminal trans-membrane™ domain, with the N-terminus intracellular. TMPred was used to assess the membrane topology of the sesorin protein. The strongly preferred model predicted only 1 transmembrane domain (score 533, considered significant if >500) located between aa 4 and 22, with the N-terminus located inside the cell. The result of this search is set out at FIG. 2.

SignalP, an established signal peptide prediction tool, indicated that the sensorin protein has a signal peptide that is cleaved after the first 17 amino acids. Sensorin may be compact and globular in shape.

Further aspects of sensorin function at the biochemical level may be determined by those skilled in the art using standard techniques. It is clear from the instant invention that sensorin is a survival factor for peripheral nerves, as the absence of this gene in humans leads to the neuropathic condition of HSAN II.

Expression: Based on ESTs, the HSN2 gene is expressed in the sympathetic trunk, kidney, testis, fetal liver spleen, head-neck tumor, germ cell tumors, muscle, uterus, retina, whole eye, and CNS (multiple sclerosis lesions). SAGE expression was detected in pancreatic adenocarcinoma, brain astrocytoma, brain glioblastoma, colon adenocarcinoma, ependymoma and normal vascular endothelial cells. There is no GNF data. Therefore the HSN2 gene appears to be widely expressed in many tissues, but at extremely low levels. HSN2 may be expressed in peripheral sensory neurons or supporting Schwann cells, which usually make up only a small percentage of any given tissue's mass.

It is recognized that those skilled in the art may prefer to use forms of HSN2 or sensorin corresponding to the sequences disclosed herein, although not necessarily the same. For example, screening assays may utilize HSN2 or sensorin from a different organism, preferably a vertebrate, and most preferably from a mammalian species. The shared technical features of these forms of HSN2 or sensorin, are that, when expressed, they have similar biological activity, and that they share functional similarity with HSN2 or sensorin, as the case may be, such as may be determined by those skilled in the art. Thus the invention encompasses the use of, including but not limited to, sheep, dog, rat, mouse or horse HSN2 or sensorin, for the same purposes as set out more specifically herein for human HSN2 or sensorin. The HSN2 gene and/or sensorin polypeptide according to the invention may also be obtained from other mammalian species, other vertebrates, invertebrates and microorganisms based on the disclosure herein.

Because of the processing that may take place in transforming the initial RNA transcript into the final mRNA, the sequences disclosed herein may represent less than the full genomic sequence. They may also represent sequences derived from alternate splicing of exons. Consequently, the genes present in the cell (and representing the genomic sequences) and the sequences disclosed herein, which are mostly cDNA sequences, may be identical or may be such that the cDNAs contain less than the full genomic sequence. Such genes and cDNA sequences are still considered corresponding sequences because they both encode similar RNA sequences. Thus, by way of non-limiting example only, a gene that encodes an RNA transcript, which is then processed into a shorter mRNA, is deemed to encode both such RNAs and therefore encodes an RNA corresponding to an HSN2 sequence as disclosed herein. Thus, the sequences disclosed herein correspond to genes contained in the cells and are used to determine relative levels of expression because they represent the same sequences or are complementary to RNAs encoded by these genes. Such genes also include different alleles and splice variants that may occur in the cells used in the processes of the invention.

Method of Treatment Using HSN2 as a Therapeutic Target.

The discovery that mutations in HSN2 relate to clearly definable physiological outcomes in humans (namely HSAN II) establishes a clear function for this protein, and now allows the inventors to establish, for the first time, that HSN2 Gene and sensorin are useful as therapeutic targets in humans for the treatment of pain, neuropathy and related disorders. Standard industrial processes are available to those skilled in the art to confirm the identity of the therapeutic agents which modulate the activity of the gene or protein, many of which are set out below.

In accordance with the foregoing, the present invention relates to a method for treating a disorder comprising administering to a person in need of such treatment an effective amount of a selective sensorin agonist or antagonist, or a pharmaceutically acceptable salt thereof, or a pharmaceutical composition containing either entity.

In a preferred embodiment, said disorder is pain, neuropathy or a related disorder. In a further preferred embodiment, said administering is by oral or intravenous means.

Identification of Therapeutic Agents.

The present invention also readily affords different means for identification of agents for treating pain, neuropathy and related disorders according to their ability to modulate the activity of HSN2 or its protein. Such means involve testing libraries of chemical compounds, either one at a time or in combinations, in an assay format which is designed to measure a biological activity related to HSN2 or sensorin, the protein encoded by HSN2. Those library compounds that modulate the biological activity in the desired fashion are thereby identified as therapeutic agents of the invention.

Exemplary assay methods useful for the identification of such compounds are detailed herein, although those skilled in the art will be aware of alternative means. In a first step, compounds are sequentially tested against the assay to determine whether they influence a measurable biological activity of the assay.

Assays may be based one or more of the diverse measurable biological activities of a gene or polypeptide corresponding to HSN2 or sensorin. Relating to the purified sensorin protein, sensorin activity includes, but is not limited to, all those biological processes, interactions, binding behavior, binding-activity relationships, pKa, pD, enzyme kinetics, stability, and functional assessments of the protein. Relating to sensorin activity in cell fractions, reconstituted cell fractions or whole cells, these activities include, but are not limited the rate at which sensorin performs any measurable biological characteristic and all measurable consequences of these effects, including cell growth, development or behavior and other direct or indirect effects of sensorin activity. Relating to HSN2 genes and transcription, HSN2 activity includes the rate, scale or scope of transcription of genomic DNA to generate RNA; the effect of regulatory proteins on such transcription, the effect of modulators of such regulatory proteins on such transcription; plus the stability and behavior of mRNA transcripts, post-transcription processing, mRNA amounts and turnover, and all measurements of the rate or amount of expression and translation of the mRNA into polypeptide sequences. Relating to HSN2 activity or sensorin activity in organisms, this includes but is not limited biological activities which are identified by their absence or deficiency in disease processes or disorders caused by aberrant HSN2 or sensorin biological activity in those organisms. Broadly speaking, HSN2 biological activity or sensorin biological activity can be determined by all these and other means for analyzing biological properties of proteins and genes that are known in the art.

The invention therefore provides numerous assays which measure an activity of HSN2 or sensorin and are useful for the testing of chemical compounds to identify which ones affect such activity.

In one aspect, the present invention relates to a method for identifying an agent that modulates the activity of the HSN2 gene, comprising:

a) contacting a test compound with a cell that expresses an HSN2 gene;

b) determining a change in the expression of said gene as a result of said contacting,

wherein said determined change in expression of the gene indicates modulation, thereby identifying the test compound as an agent that modulates the activity of an HSN2 gene.

In a preferred embodiment of such method, HSN2 is a mammalian HSN2 gene, most preferably where the mammal is mouse, rat or human. Also preferred is where determined modulation is a decrease in expression and/or where the gene is present in a cell, preferably a mammalian cell, preferably a nervous system cell, such as a neuron. Such cells may include a recombinant cell, especially one genetically engineered to contain and express the HSN2 gene. In a preferred embodiment, said gene has the sequence of SEQ ID NO: 1.

In other embodiments, the gene comprises a polynucleotide corresponding to a polynucleotide having a nucleotide sequence selected from the group consisting of SEQ ID NO: 1, 7 and 9.

In another aspect, the present invention relates to a method for identifying an agent that modulates HSN2 gene, comprising:

(a) contacting a test compound with a genetic construct comprising a reporter gene operably linked to an HSN2 promoter under conditions where the reporter gene is expressed;

(b) determining a change in-expression of the reporter gene as a result of said contacting,

wherein a determined change in expression indicates modulation, thereby identifying the test compound as an agent that modulates HSN2 gene.

In a preferred embodiment thereof, the modulation is a decrease in expression of said reporter gene. Normally the reporter gene is some readily measured gene and is not HSN2 but the HSN2 gene, with promoter sequence, may be used and still lie within the contemplation of the invention, even though the gene is not contained within a cell. Also preferred is where the HSN2 promoter is a mammalian HSN2 promoter, especially where the mammal is mouse, rat or human. Most preferred is where the promoter has the nucleotide sequence of SEQ ID NO: 6 and/or where the genetic construct is present in a cell, preferably a mammalian cell, such as a nervous system cell, preferably a neuron. Of course, such construct will normally be present in a recombinant cell.

The present invention also contemplates a method for identifying an agent that modulates the activity of an HSN2-encoded protein, comprising:

a) contacting a test compound with an HSN2-encoded polypeptide under conditions where said polypeptide is active; and

b) determining a change in the activity of said polypeptide as a result of said contacting;

wherein said determined change in activity indicates modulation, thereby identifying the test compound as an agent that modulates the activity of HSN2-encoded protein (such as sensorin).

In a preferred embodiment thereof, the HSN2-encoded polypeptide is a mammalian HSN2-encoded polypeptide, preferably where the mammal is mouse, rat or human. Also preferred is where the determined modulation is a decrease in activity of the polypeptide.

Also preferred is where the polypeptide is present in a mammalian cell, especially where this has been engineered to contain or express said polypeptide, such as by genetic engineering. In one such embodiment, the recombinant cell does not express said polypeptide absent said engineering. Also preferred is where the cell is a cell of the nervous system, especially a neuron, most especially a neuron involved in pain response.

Also preferred is where the polypeptide is encoded by a polynucleotide having a sequence selected from the group consisting of SEQ ID NO: 1, 7, and 9, especially where the polypeptide comprises an amino acid sequence of SEQ ID NO: 2, 8, 10 or 11.

In a preferred embodiment, the observed change in activity in step (b) is a decrease in activity and is the result of binding to said polypeptide by said chemical agent of step (b). Also preferred is where the agents are useful for treating pain, neuropathy or a related disorder.

In other preferred embodiments, the polypeptide is part of an intact cell, and the present invention specifically contemplates embodiments in which the cell is engineered by other than genetic engineering, such as where the activity of a polypeptide is to be enhanced and the cell has been engineered to contain, or have on its surface, said polypeptide but wherein the polypeptide is present due to physical insertion of the polypeptide into the membrane or cytoplasm of the cell and not through expression of a gene contained in the cell. Methods well known in the art, such as use of polyethylene glycol, viruses, and the like, are available to effect such insertions and the details of such procedures need not be further described herein.

In one preferred embodiment of such method, the polypeptide is a polypeptide that reacts with and/or binds to, an antibody that binds to, or is specific for, a polypeptide having an amino acid sequence at least 95% identical to, more preferably at least 98% identical to, the sequence of SEQ ID NO: 2 and where any difference in amino acid sequence is due only to conservative amino acid substitutions. In an especially preferred embodiment, the polypeptide has the amino acid sequence of SEQ ID NO: 2.

The sensorin assays of the invention may employ compound screening technology such as (but not limited to) the ability of various dyes to change color in response to changes in assay conditions resulting from the activity of the polypeptides. Compound screening assays can also be based upon the ability of test compounds to modulate the interaction of the target peptide (sensorin) and known interacting proteins. Such interacting proteins can be identified by a variety of methods known in the art, including, for example, radioimmunoprecipitation, co-immunoprecipitation, co-purification, and yeast two-hybrid screening. Such interactions can be further assayed by means including but not limited to fluorescence polarization or scintillation proximity methods.

Agents that have the effect of modulate the half-life of sensorin in cells would also act to induce decreased sensitivity to pain and thereby achieve analgesia. By way of non-limiting example, cells expressing a wild-type sensorin are transiently metabolically labeled during translation, contacted with a candidate compound, and the half-life of the polypeptide is determined using standard techniques. Compounds that modulate the half-life of the polypeptide are useful compounds in the present invention.

In one such assay for which the polypeptides encoded by genes disclosed herein are useful, the polypeptide (or a polypeptide fragment thereof or an epitope-tagged form or fragment thereof) is bound to a suitable support (e.g., nitrocellulose or an antibody or a metal agarose column in the case of, for example, a his-tagged form of said polypeptide). Binding to the support is preferably done under conditions that allow proteins associated with the polypeptide to remain associated with it. Such conditions may include use of buffers that minimize interference with protein-protein interactions. If desired, other proteins (e.g., a cell lysate) are added, and allowed time to associate with the polypeptide. The immobilized polypeptide is then washed to remove proteins or other cell constituents that may be non-specifically associated with it the polypeptide or the support. The immobilized polypeptide can then be used for multiple purposes. In a compound screening embodiment, compounds can be tested for their ability to interfere with interactions between sensorin and other bound molecules (which are presumably sensorin interacting proteins). Compounds which can successfully displace interacting proteins are thereby identified as sensorin modulating agents of the invention.

In an alternative embodiment designed to identify sensorin interacting proteins, the immobilized polypeptide is dissociated from its support, and proteins bound to it are released (for example, by heating), or, alternatively, associated proteins are released from the polypeptide without releasing the latter polypeptide from the support. The released proteins and other cell constituents can be analyzed, for example, by SDS-PAGE gel electrophoresis, Western blotting and detection with specific antibodies, phospho-amino acid analysis, protease digestion, protein sequencing, or isoelectric focusing. Normal and mutant forms of such polypeptide can be employed in these assays to gain additional information about which part of the polypeptide a given factor is binding to. In addition, when incompletely purified polypeptide is employed, comparison of the normal and mutant forms of the protein can be used to help distinguish true binding proteins. Such an assay can be performed using a purified or semipurified protein or other molecule that is known to interact with a polypeptide encoded by an HSN2 polynucleotide.

This assay may include the following steps.

1. Harvest the encoded polypeptide and couple a suitable fluorescent label to it,

2. Label an interacting protein (or other molecule) with a second, different fluorescent label. Use dyes that will produce different quenching patterns when they are in close proximity to each other versus when they are physically separate (i.e., dyes that quench each other when they are close together but fluoresce when they are not in close proximity);

3. Expose the interacting molecule to the immobilized polypeptide in the presence or absence of a compound being tested for its ability to interfere with an interaction between the two; and

4. Collect fluorescent readout data.

An alternative assay for such protein interaction is the Fluorescent Resonance Energy Transfer (FRET) assay. This assay can be performed as follows.

1. Provide the encoded protein or a suitable polypeptide fragment thereof and couple a suitable FRET donor (e.g., nitro-benzoxadiazole (NBD)) to it;

2. Label an interacting protein (or other molecule) with a FRET acceptor (e.g., rhodamine);

3. Expose the acceptor-labeled interacting molecule to the donor-labeled polypeptide in the presence or absence of a compound being tested for its ability to interfere with an interaction between the two; and

4. Measure fluorescence resonance energy transfer.

Quenching and FRET assays are related. Either one can be applied in a given case, depending on which pair of fluorophores is used in the assay.

Additionally, drug screening assays can also be based upon polypeptide functions deduced upon antisense interference with the gene function. Intracellular localization of pain-related polypeptides, or effects which occur upon a change in intracellular localization of such proteins, can also be used as an assay for drug screening.

In accordance with the foregoing, the present invention provides the amino acid sequence of a protein, designated sensorin or HSN2 Polypeptide, that is found in neuronal cells (for example, SEQ ID NO: 2 from humans) and which is associated with hereditary transmission of insensitivity to pain and neuropathy. In addition, several mutations, have been found in this sequence derived from individuals found to have such indifference to pain. Thus, agents that mimic the phenotypic effects of this mutations, such as aberrant protein structure and decreased, or absent, function represent candidate compounds for evaluation as therapeutic agents of the invention.

Relating to expression assays, in one aspect the present invention relates to a method for identifying an agent that modulates the activity of a polynucleotide whose expression contributes to pain sensation, comprising:

a) contacting under physiological conditions a chemical agent with a polynucleotide corresponding to the promoter region of HSN2 (in a preferred embodiment, the promoter has the sequence of SEQ ID NO: 6); and

b) detecting a change in the expression of said polynucleotide as a result of said contacting;

thereby identifying an agent that modulates said polynucleotide or gene activity.

Such modulation is preferably a decrease or an increase in expression. In preferred embodiments, such expression is measured by measuring the amount of an expression product. In a convenient embodiment, the promoter region of HSN2 is operably linked to a reporter gene, that is, a gene which whose expression is conveniently measured (for example, reporter genes such as Green Fluorescent Protein, luciferase, chloramphenicol acetyl-transferase (CAT), and the like).

In preferred embodiments, the polynucleotide whose expression is to be measured or monitored is present in an intact cell, preferably a mammalian cell, most preferably a neuronal cell. In additional preferred embodiments, such an intact cell is a cell that has been engineered to comprise said polynucleotide, such as by genetic engineering, most preferably wherein the cell does not express the subject gene or polynucleotide absent having been engineered to do so.

In accordance with the disclosure herein, upstream untranslated regions and promoter regions of HSN2 are readily obtained from SEQ ID No. 12 and other publicly retrievable sequence databases. Such genomic or untranslated regions may be included in plasmids comprising the identified gene, such as in assays to identify compounds which modulate expression thereof. In one such assay, the upstream genomic region is ligated to a reporter gene, and incorporated into an expression plasmid. The plasmid is transfected into a cell, and the recombinant cell is exposed to test compound(s). Those compounds which increase or decrease the expression of the reporter gene are then modulators of the gene/protein, and are considered therapeutic agents of the invention.

The invention also claims recombinant cells engineered to express a polynucleotide or polypeptide as disclosed herein. The gene disclosed herein as being involved in HSAN II in an animal can be used, or a fragment thereof can be used, as a tool to express a protein, where such genes encode a protein, in an appropriate cell in vitro, or can be cloned into expression vectors which can be used to produce large enough amounts of protein to use in in vitro assays for drug screening. Alternatively, the expression construct may employ the genomic promoter region of HSN2 and link it to a gene, such as a reporter gene, whose expression level is easily measured. Expression systems which may be employed include baculovirus, herpes virus, adenovirus, adeno-associated virus, bacterial systems, and eukaryotic systems such as CHO cells. Naked DNA and DNA-liposome complexes can also be used. The invention thus claims recombinant cell lines containing a heterologous HSN2 gene.

For general molecular biology procedures useful in practicing the present invention, a number of standard references acre available that contain procedures well known in the art of molecular biology and genetic engineering and which procedures need not be further described herein. Useful references include Sambrook, et al., Molecular Cloning: A Laboratory Manual, Second Edition, Cold Spring Harbor, N.Y., (1989), Wu et al, Methods in Gene Biotechnology (CRC Press, New York, N.Y., 1997), and Recombinant Gene Expression Protocols, in Methods in Molecular Biology, Vol. 62, (Tuan, ed., Humana Press, Totowa, N.J., 1997), the disclosures of which are hereby incorporated by reference.

Such recombinant cells may be used in expression assays for analyzing the levels of expression of HSN2 or a suitable reporter gene after contacting said cells with agents that may have analgesic properties. The levels of gene expression can be quantified by Northern blot analysis or RT-PCR, or, alternatively, by measuring the amount of protein produced, by one of a number of methods known in the art, or by measuring the levels of biological activity of polypeptides encoded thereby or other genes. In this way, the gene expression can serve as a marker, indicative of the physiological response of the cells to the agent. Accordingly, this response state may be determined before, and at various points during, treatment of the individual with the agent.

Recombinant cell lines are also preferred for the preparation of purified protein, if a purified protein assay is desired. Those skilled in the art are capable of producing recombinant cell lines and extracting protein fractions containing highly purified proteins. These samples can be used in a variety of binding assays to identify compounds which interact with the proteins. Compounds that interact are therapeutic agents of the invention, or analogs thereof.

Target selectivity is an important aspect of the development of therapeutic agents. The present invention specifically contemplates the identification of chemical agents, especially small organic molecules, that inhibit the expression of HSN2, as defined herein, or the activity of the sensorin polypeptide (such as SEQ ID NO: 2 from humans) encoded thereby, with high specificity and that have little or no effect on other genes and/or polypeptides.

Thus, in one such preferred embodiment, the methods disclosed herein for identifying an agent that modulates, preferably inhibits, expression of a gene corresponding to the HSAN gene, preferably having the sequence of SEQ ID NO: 1 from humans, or on the activity of a polypeptide encoded thereby, comprises first identifying such agent and then testing such agent for effects on expression or activity of at least one other gene or polypeptide, preferably a gene or polypeptide with important physiological consequences that are preferably not disturbed by therapeutic intervention, and demonstrating little or no effect.

In another aspect, the invention provides a method for computationally identifying a compound of the invention. The method involves (a) determining the active site of a-sensorin protein (i.e. through X-Ray crystallography or other techniques); and (b) through computational modeling, identifying a compound which interacts with the active site, thereby identifying a compound, or its analog, as a compound which is useful for modulating the activity of such a polypeptide. This process is sometimes referred to as in silico screening. Sophisticated software for testing the probability of test compounds to interact with the target protein, which can test tens of millions of computer generated compounds, is available to those skilled in the art.

Potential therapeutic compounds identified using the methods of the invention are usually tested in animal model systems to confirm the putative efficacy. Thus, in a further aspect, the present invention relates to a method for identifying an analgesic agent, comprising:

(a) administering to an animal an agent found to modulate HSN2 gene or protein activity, and

b) determining in said animal a decrease in response to a pain stimulus as a result of said administering,

wherein a determined decrease in response to said pain stimulus indicates analgesic activity, thereby identifying said agent an analgesic agent.

Preferably, the animal is a mammal, such as a human being. In specific embodiments, the pain stimulus is a heat stimulus and reaction or sensitivity to hot and/or cold may be measured. In another embodiment, an electrical stimulus may be used. In all cases, the stimulus may be represented as a sharp or dull sensation. In some cases, the animal may otherwise react normally to such stimulus so that a decrease in normal response due to the test agent is being measured whereas in other cases the animal may initially possess a heightened sensitivity to the stimulus prior to administering the test agent. In all cases, observation of an analgesic effect need not necessarily involve a reduced sensitivity or response to pain but may involve simply a reduced sensation of a particular stimulus. The analgesics identified by the methods of the invention may induce general analgesia in an animal or may have more localized analgesic or anesthetic effects.

In a further aspect, the present invention relates to a method for treating a condition in an animal afflicted with a source of chronic pain comprising administering to said animal an effective amount of an analgesic agent first identified by an assay method of the invention. Preferably, said animal is a human patient, such as a patient afflicted with a chronic ailment, such as a cancerous condition.

The screening assays of the invention thus simplify the evaluation, identification and development of therapeutic agents for the treatment of pain, neuropathy and related disorders.

The invention also includes antibodies and immuno-reactive substances which target, interact with or bind to sensorin or epitopes thereof. Polypeptides encoded by the polynucleotides disclosed herein can be used as an antigen to raise antibodies, including monoclonal antibodies. Such antibodies will be useful for a wide variety of purposes such as for therapeutic agents, in functional studies, in drug screening assays, in clinical trials and for diagnostics.

For example, in drug screening assays or in clinical trials, the effectiveness of an agent determined by a screening assay as described herein to increase or decrease gene expression, protein levels, or biological activity can be monitored using antibodies specific for sensorin. Alternatively, the effectiveness of an agent determined by a screening assay to modulate expression of HSN2, as well as structurally and functionally related genes, including genes with high homology thereto, and including protein levels, or biological activity can be monitored in clinical trials of subjects exhibiting decreased altered gene expression, protein levels, or biological activity. In such clinical trials, the expression or activity of the genes or polypeptides disclosed herein and, preferably, other genes that have been implicated in, for example, congenital resistance to pain stimuli, can be used to ascertain the effectiveness of a particular analgesic drug.

In a preferred embodiment, the present invention provides a method for monitoring the effectiveness of treatment of a subject with an agent (e.g., an agonist, antagonist, peptidomimetic, protein, peptide, antibody, nucleic acid, small molecule, or other drug candidate identified by the screening assays described herein) including the steps of (i) determining that a patient exhibits discomfort due to a disease or disorder that causes some type of painful stimulus; (ii) administering an effective amount of an agent identified using one of the screening assays disclosed herein; (iii) ascertaining a reduction to pain or other stimuli following said administration and (vi) altering the administration of the agent to the subject accordingly. For example, increased administration of the agent may be desirable to decrease the expression or activity of gene or encoded polypeptide, i.e., to increase the effectiveness of the agent.

Where the patient is non-human, biopsy samples can be taken to show a decrease in gene expression, such as by measuring levels of protein, mRNA, or genomic DNA post-administration samples and comparing the level of expression or activity of said protein, mRNA, or genomic DNA in the pre-administration sample with that of the corresponding post administration sample or samples, thereby shoving the effects of drug administration on one or more of the genes disclosed herein and concomitant reduction in pain response and/or sensitivity.

Candidate modulators may be purified (or substantially purified) molecules or may be one component of a mixture of compounds (e.g., an extract or supernatant obtained from cells). In a mixed compound assay, gene expression is tested against progressively smaller subsets of the candidate compound pool (e.g., produced by standard purification techniques, e.g., HPLC or FPLC) until a single compound or minimal compound mixture is demonstrated to modulate gene or protein activity or expression in a manner having analgesic effects.

Specific compounds which will modulate HSN2 gene expression, gene transcript levels, and protein levels, in a cell include antisense nucleic acids, ribozymes and other nucleic acid compositions which specifically hybridize with HSN2 (including exons or introns of such genes, promoters, 3′-tails, and the like). These specific compounds are compounds of the invention, and are useful for treating the diseases discussed previously. Design and manufacturing of such compounds are well known to those skilled in the art.

Specific compounds which modulate the activity of HSN2, more specifically of sensorin, include antibodies (polyclonal or monoclonal) which bind specifically to an epitope of said polypeptide. These antibody compositions are compounds of the invention, and are useful for treating the diseases previously discussed. Design and manufacturing of such compounds are well known to those skilled in the art.

Specific compounds which modulate the activity of HSN2, or its encoded protein, the body include gene therapy vectors comprising all or a part of the HSN2 gene sequence or a mutant HSN2 sequence. As is well known to those skilled in the art gene therapy allows the delivery of HSN2 in an organism to cells where it is taken up and expressed, thus changing the level or amount of sensorin protein in such cell. These vectors thereby modulate the activity of HSN2 or sensorin in the body and are useful for the therapeutic indications disclosed herein.

Specific compounds which modulate the activity of HSN2 or sensorin in the body include small organic molecules. Such compounds may be naturally occurring, or they may be synthetic. Collections and combinatorial libraries of such compounds are widely available from commercial sources. As know to those skilled in the art, a screening assay, such as the assays disclosed in the instant specification, can be easily adapted to identify therapeutic agents which have the desired HSN2 or sensorin modulating ability. Agonists, antagonists, or mimetics found to be effective at reducing response to pain stimuli may be confirmed as useful in animal models (for example, mice, chimpanzees, etc.). In other embodiments, treatment with a compound of the invention may be combined with other analgesics to achieve a combined, possibly even synergistic, effect.

Purified or semi-purified HSN2-encoded protein, or sensorin, and any biochemically modified versions thereof, are themselves therapeutic agents of the invention. Recombinant or non-recombinant forms of these proteins or fragments can be administered to persons in need thereof for the treatment of disorders, such as disorders disclosed herein. Preferably, such agents are administered in a pharmaceutically acceptable carrier. Those skilled in the art are familiar with techniques for generating such agents, and for determining conditions of administration.

Lead Optimization and Analog Development and Selection

In general, novel drugs having analgesic or anesthetic properties, and which modulate sensorin activity, are identified from libraries, possibly large libraries, of both natural product or synthetic (or semi-synthetic) extracts or chemical libraries according to methods known in the art. Those skilled in the field or drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

De-replication(e.g., taxonomic dereplication, biological dereplication, and chemical dereplication, or any combination thereof) or the elimination of replicates or repeats of materials already known for their analgesic and/or anesthetic activities may be employed whenever possible.

When a crude extract is found to have analgesic and/or anesthetic activities, or both, further fractionation of the positive lead extract is possible to isolate chemical constituent responsible for the observed effect. Thus, the goal of the extraction, fractionation, and purification process is the careful characterization and identification of a chemical entity within the crude extract having such analgesic and/or anesthetic activities. The same assays described herein for the detection of activities in mixtures of compounds can be used to purify the active component and to test derivatives thereof. Methods of fractionation and purification of such heterogeneous extracts are known in the art. If desired, compounds shown to be useful agents for the treatment of pathogenicity are chemically modified according to methods known in the art. Compounds identified as being of therapeutic value are subsequently analyzed using any standard animal model of diabetes or obesity known in the art.

In general, these screening methods provide a ready means for selecting either natural product extracts or synthetic compounds of interest from a large population (i.e. a chemical library, for example, one produced by combinatorial means) which are further evaluated and condensed to a few active core structures. Multiple analogs of such core structures may be developed and tested to identify those preferred analogs which have improved characteristics as therapeutic agents.

Improved analogs may also include compounds with improved stability, biodistribution, pharmacokinetics or other desirable features for therapeutic agents which are not directly related to modulation of the therapeutic target. In a preferred embodiment, the improved analog of the invention is effectively delivered, either by physiological means or assisted means, to cells of the body expressing the sensorin protein. Analog compounds are systematically screened to evaluate whether they modulate the identified biological activity and those that effectively do so are then therapeutic agents, or further analogs thereof, according to the invention.

Therapeutic Agents and Uses Thereof

The agents contemplated by the present invention are highly selective for the HSN2-encoded protein (sensorin) and administration of such an agent to a human or other animal in need thereof provides a treatment for any of the disorders exemplified by, or later found to be related to, HSAN II (and involving possible mutations of HSN2). For example, an inhibitor mimics the effects of one or more of the mutated forms of the gene as disclosed herein. Alternately, an agonist would reverse a disorder made evident in HSAN II. HSN2 and/or sensorin may also function in the development, survival or maintenance of peripheral sensory neurons, or supporting cells and thus may be useful in identifying agents that act directly to further the life and activities of such cells. Genetic or pharmacological down-regulation of this protein may lead to novel analgesics used to treat pain or other neurological symptoms, whereas up-regulation may lead to novel neuronal survival agents which may be useful in treating a number of neurodegenerative disorders of both the CNS and PNS. As such, the agent identified according to this invention is a treatment for pain, neuropathy or a related disorder.

Disorders which may be related to aberrant HSN2 or sensorin activity include pain, for example inflammatory pain, a neuralgia, a nerve entrapment syndrome, and pain associated with a musculoskeletal disorder; neuropathies, for example peripheral, diabetic, autonomic, sensory or motor neuropathy; control of sweating; autonomic dysfunction such as control of blood-pressure, swallowing, posture, hypertension, hypotension, GI—esophageal dysmotility, impotence, apnea, absence of lacrimation; Riley-Day Syndrome, temperature control, hypertonia; delayed gastric emptying, diarrhea, vomiting, neurological disorders such as seizure, migraine, tremor, anxiety, depression, hyperphagia and associated obesity, bipolar disorder, Parkinson's, Huntington's, spinal cord injury, multiple sclerosis, traumatic brain injury, stroke, neurodegenerative disorders such as Alzheimer's Disease and ALS, and the like.

A variety of diseases are also treatable based on sensorin's possible role as a novel neurotrophic growth factor in the nervous system. Sensorin's amino acid sequence is about twice the length of most other known neurotrophic factors, and may represent a proform that is proteolytically cleaved at positions other than at the signal peptide cleavage site. Like other neurotrophic factors, sensorin may be expressed at low levels.

Neurotrophic factors are essential for the development of the nervous system. They exert a diverse set of influences including neuronal cell survival, axonal and dendritic growth and guidance, synaptic structure and connections, neurotransmitter release, long-term potentiation and synaptic plasticity. Alterations in neurotrophic factor levels can influence cell survival and apoptosis during development, myelination, regeneration, pain, aggression, depression, substance abuse, anxiety, hyperphagia, memory acquisition and retention and loss of the peripheral nervous system. It is thus suggested that administration of sensorin protein, or fragments thereof, or modulators of sensorin or HSN2 activity are useful for the treatment of these diseases.

Those skilled in the art are familiar with the necessary steps for pre-clinical and human clinical trials which are used to establish efficacy and safety of the new chemical entities and compounds first identified by the invention for use in treating the diseases mentioned herein.

Compounds first identified as useful in reducing sensitivity to pain stimuli using one or more of the assays of the invention may be administered with a pharmaceutically-acceptable diluent, carrier, or excipient, in unit dosage form. Conventional pharmaceutical practice may be employed to provide suitable formulations or compositions to administer such compositions to patients. Although oral administration is preferred, any appropriate route of administration may be employed, for example, intravenous, parenteral, subcutaneous, intramuscular, intracranial, intraorbital, ophthalmic, intraventricular, intracapsular, intraspinal, intrathecal, epidural, intracisternal, intraperitoneal, intranasal, or aerosol administration. Therapeutic formulations may be in the form of liquid solutions or suspension; for oral administration, formulations may be in the form of tablets or capsules; and for intranasal formulations, in the form of powders, nasal drops, or aerosols.

Methods well known in the art for making formulations are found in, for example, Remington: The Science and Practice of Pharmacy, (19th ed.) ed. A. R. Gennaro AR., 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for agonists of the invention include ethylenevinyl acetate copolymer particles, osmotic pumps, implantable infusion systems, and liposomes. Formulations for inhalation may contain excipients, or example, lactose, or may be aqueous solutions containing, for example, polyoxyethylene-9-lauryl ether, glycocholate and deoxycholate, or may be oily solutions for administration in the form of nasal drops, or as a gel.

Combination therapies are also contemplated by the inventors. An analgesic agent identified by one of the screening methods disclosed herein may be administered along with another agent intended to treat a coincident conditions, such as where analgesic and antitumor agents are given together or contemporaneously.

The present invention also relates to a process that comprises a method for producing a product, such as by generating test data to facilitate identification of such product, comprising identifying an agent according to one of the disclosed processes for identifying such an agent (i.e., the therapeutic agents identified according to the assay procedures disclosed herein) wherein said product is the data collected with respect to said agent as a result of said identification process, or assay, and wherein said data is sufficient to convey the chemical character and/or structure and/or properties of said agent. For example, the present invention specifically contemplates a situation whereby a user of an assay of the invention may use the assay to screen for compounds having the desired enzyme modulating activity and, having identified the compound, then conveys that information (i.e., information as to structure, dosage, etc) to another user who then utilizes the information to reproduce the agent and administer it for therapeutic or research purposes according to the invention. For example, the user of the assay (user 1) may screen a number of test compounds without knowing the structure or identity of the compounds (such as where a number of code numbers are used the first user is simply given samples labeled with said code numbers) and, after, performing the screening process, using one or more assay processes of the present invention, then imparts to a second user (user 2), verbally or in writing or some equivalent fashion, sufficient information to identify the compounds having a particular modulating activity (for example, the code number with the corresponding results). This transmission of information from user 1 to user 2 is specifically contemplated by the present invention.

In accordance with the foregoing, the present invention encompasses a method for producing test data with respect to the gene modulating activity of a compound comprising:

(a) contacting a test compound with a HSN2 gene under conditions where said gene is being expressed;

b) determining a change in the expression of said gene as a result of said contacting, and

(c) producing test data with respect to the gene modulating activity of said test compound based on a change in the expression of the determined gene as a result of said contacting.

Diagnostics and Pharmacogenomics

In a further embodiment; the invention relates to diagnostic and pharmacogenomic compounds, kits and methods. This aspect relates to analysis HSN2 for the diagnosis of insensitivity or indifference to pain, other pain disorder, or in the selection of a therapeutic agent for a patient (i.e. pharmacogenomics).

For example, nucleic acid analysis can be used to identify the HSN2 mutations disclosed herein, thus confirming the diagnosis of HSAN II. Many nucleic acid diagnostic techniques are well known to those skilled in the art. Such techniques include DNA sequencing, hybridization probing, single stranded conformational analysis, PCR based techniques such as mismatch amplification, and myriad other well known methods. All such analysis can be performed on a small sample of blood, saliva, urine or other tissue provided by the patient.

Alternatively, protein based analyses, such as antibody based assays (Elisa, Radioimmunoassay and the like) can be employed to identify the expression, amount or presence or absence of a mutant protein (such as sensorin encoded by a mutant HSN2), such as those disclosed herein.

Gene expression, both comparable and absolute, as well as biological activity, and mutational analysis can each serve as a diagnostic tool for pain or neuropathic disorders; thus determination of the amount of HSN2 mRNA can be used to diagnose the presence or absence of a mutation correlated with such pain or neuropathic disorder.

Pharmacogenomics deals with clinically significant hereditary variations in the response to drugs due to altered drug disposition and abnormal action in affected persons (Eichelbaum, M., Clin. Exp. Pharmacol. Physiol., 23:983-985, 1996; Linder, M. W., Clin. Chem., 43:254-266, 1997). In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). Altered drug action may occur in a patient having a polymorphism (e.g., an single nucleotide polymorphism or SNP) in promoter, intronic, or exonic sequences of HSN2. Thus by determining the presence and prevalence of polymorphisms in HSN2 in an individual, one may predict a patients response to a particular therapeutic agent.

This pharmacogenomic analysis can lead to the tailoring of drug treatments according to patient genotype, including prediction of side effects upon administration of therapeutic agents, particularly therapeutic agents for treating disorders disclosed in this specification. Pharmacogenomics allows for the selection of agents (e.g., drugs) for therapeutic or prophylactic treatment of an individual based on the genotype of the individual (e.g., the genotype of the individual is examined to determine the ability of the individual to respond to a particular agent).

Diagnostics employing a gene or protein corresponding to HSN2 can also be useful in selecting patients for clinical trials of a potential therapeutic agent. Patients can be stratified according to the DNA or protein sequence of HSN2 and their response to drug treatment can be evaluated. Such stratification can greatly reduce the number of patients required to establish efficacy for a potential therapeutic agent.

### EXAMPLE 1

### Identification of the Genetic Mutation Responsible for Hereditary Sensory and Autonomic Neuropathy—Type II (HSAN II)

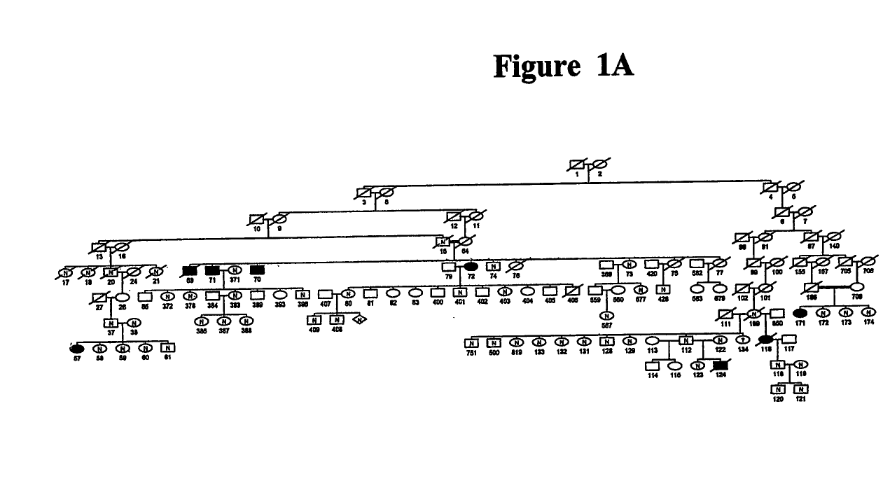

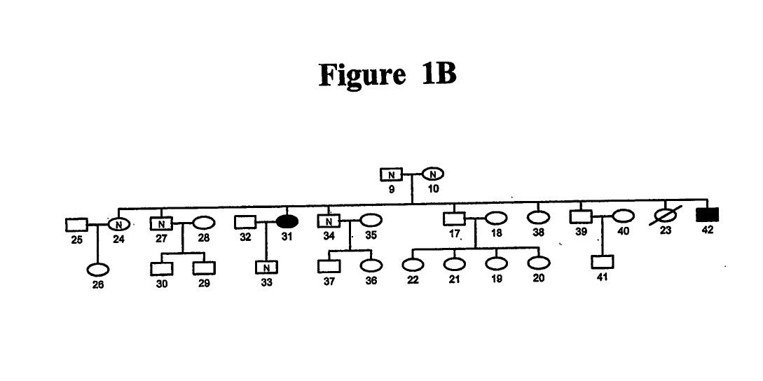

We performed a genomewide screen in two geographically isolated families, who are likely related, to map the HSAN II locus. We expanded a consanguineous multigenerational family with eight affecteds (HSAN4) from a sibship reported previously and collected a smaller family with two affecteds (HSAN3). Most of the patients live within a 100-mile radius in a geographically isolated region. In both families the mode of inheritance was autosomal recessive and affected members presented with severe and early-onset HSAN II. Sensory loss was predominantly distal, but the progression of the disorder varied, involving the trunk in some patients. Because of the common geographic origin of our subjects and the relationships established through family history, it became clear that the different degrees of sensory loss were part of a continuum of a single disease process, and both families were expected to segregate the identical mutation. Pedigrees are shown in FIG. 1. Additionally, unrelated affected samples from a different population were available, and these consisted of two affected sisters (HSAN5-301 and HSAN5-302) and another affected individual with no close family ties to the affected sisters (HSAN6-301).

Fifteen family members from HSAN4 and seven family members from HSAN3 were genotyped at 763 autosomal markers and at 48× markers. A genome-wide two-point analysis identified five regions with cumulative two-point LOD scores >2.0. Only one region contained a marker that was homozygous in the three affected siblings in HSAN4 (patients 70-72) and the two affected siblings in HSAN3 (patients 31-42). This marker, D12S352, with a positive cumulative LOD score of 2.64 at zero recombination, suggested that HSAN type II likely maps to chromosome 12p13. In addition, this was the only genomic segment where a three-marker haplotype was shared between all affecteds. Analysis of additional microsatellite markers demonstrated that all affected individuals in both families were homozygous for each marker in a 1.2 Mb common haplotype. Marker order was consistent between the Genethon and Marshfield genetic maps and the Build 30 human genome sequence assembly, suggesting that there were no gross inconsistencies or recombination hotspots. 22 markers gave significant positive cumulative LOD scores, the highest one being 8.4 for marker CA1AC005343 at zero recombination.

The core region of shared marker alleles (see Table 1) places the HSAN II locus within a region on chromosome 12p13 between markers CA1AC0021054 (forward primer: 5′-ACCATCACCTAAGGAGACAGACC (SEQ ID NO: 30) and reverse primer: 5′-TGCAACAAATGTACCACTCTGG (SEQ ID NO: 31)) and D12S1642 (forward primer: 5′-AGCTCCTAAATCCCCG (SEQ ID NO: 32), and reverse primer: 5′-GCCATGTCTATAAATACCCTG (SEQ ID NO: 33)) based on recombination breakpoints in individuals HSAN4-171 and HSAN4-124.

We also screened the additional affecteds from a different population. Individual HSAN6-301 was homozygous for all markers in the region defined in the HSAN4 family. The two affected sisters, HSAN5-301 and HSAN5-302, were not homozygous in the 12p13 region, and instead shared alleles on one chromosome with individual HSAN6-301. Alleles on the other chromosome are shared between the two sisters, suggesting that they are compound heterozygotes and that there are two founder mutations in this population. A recombination in one of the HSAN5 sisters positions the linked region above marker CA1AC005183x2.

The genomic sequence for the region defined in family HSAN4 is well defined, with a BAC contig consisting of 12 completely finished and one incomplete BAC sequences. The syntenic region in mouse is ˜900 kb in size and located on chr 6. The gene order is conserved between human and mouse.

TABLE 1
Definition of the HSAN Ii minimal candidate region

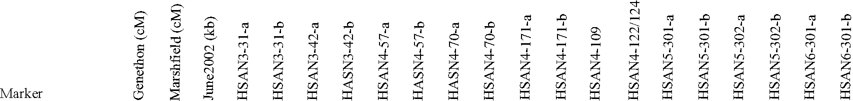

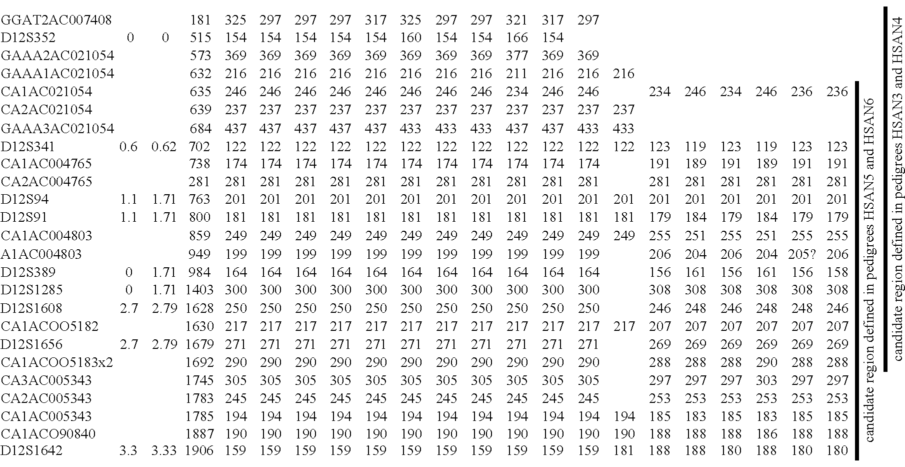

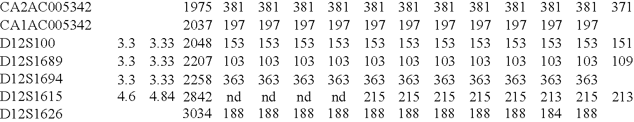

Candidate genes: A list of the genes mapping to this candidate interval was compiled based on the April 2002 freeze of the human genome assembly available from UCSC. A total of 13 known or predicted transcripts are located between markers D12S352 and D12S1615. These included NINJ2 (NM_{—}016533), FLJ31553 (AK056115), PRKWNK1 (NM_{—}018979), RAD52 (NM_{—}002879), ELKS (NM_{—}015064); PPA (BM714852), WNT5B (NM_{—}030775), FLJ21432 (BM453101), CACNA2D4 (AL137658), SLIT2L (BI754042), FLJ25180 (AK057909), FLJ31638 (AK056200) and CACNA1C (NM_{—}000719). The transcripts were categorized according to their evidence for being real genes, according to the following scheme: 1—confirmed mRNA; 2—mRNA supported by ESTs; 3—spliced ESTs with other evidence to support; 4—single spliced EST; 5—unspliced EST; 6—pseudogene not expressed. Many unspliced ESTs were found, and no serious effort was taken to label these as transcripts, given the known contamination of genomic DNA or unspliced intronic contamination within the cDNA libraries used to generate dbEST. Also, gene predictions were generally ignored unless there was independent evidence from mRNA sequence, EST, or non-human gene in which case the mRNA was used as transcript information.

Sequencing of the coding regions of these initial 13 candidate genes did not identify any relevant mutations. A more exhaustive search for candidate genes was therefore conducted based on conserved homologies between human and mouse genomic assemblies. The human genomic sequence representing the entire HSAN II candidate interval (1.2 Mb) was downloaded from the Human Genome Browser website (genome.ucsc.edu/) with case toggled to highlight the mouse translated BLAT track (which represents regions showing significant protein homology between human and mouse). A similar human sequence showing the translated Tetraodon BLAT track was also downloaded. These sequences were assembled into a contig, to which all exons of previously identified candidate genes were added. Then the contig was scanned for novel conserved fragments, roughly >80% conserved over >100 bp. This identified 64 novel fragments, which were then tested for functional homolgy using: 1) BLASTn against the nr database, 2) BLASTn against the dbEST, and 3) BLASTx against the nr database available from the NCBI website (at www.ncbi.nlm.nih.gov/BLAST/). The HSN2 gene contained the only novel well-conserved ORF identified in this screen.

Assembly of HSN2 cDNA contig and transcripts: A total of 21 human EST sequences (from 8 different cDNA libraries), including BG428619, BF522762, and N98701, and a genomic sequence from BAC AC004765, were assembled into a contig. A putative cDNA sequence was derived from the genomic sequence, with ends based on human. ESTs (SEQ ID NO. 1). The start ATG is immediately preceded by a stop TGA codon, and there is no further indication of a conserved splicing site upstream of the ORF, suggesting that this gene consists of a single unspliced exon. The identified open reading frame (ORF) encoding a peptide of 434 aa (SEQ ID NO. 2) was used to identify the mouse, rat, and fish orthologs from their respective genome assemblies. A consensus genomic sequence was downloaded from the UCSC website (SEQ ID NO. 12). The HSN2 gene maps to intron 8 of the PRKWNK1 gene, and consists of a single exon that is transcribed from the same strand as PRKWNK1.

To screen the ORF for nucleic acid mutations, three separate amplicons were designed (XH18x01a, XH18x01b, and XH18x01c). PCR primers (XGR0720 to XGR0725) were designed using PrimerSelect (DNASTAR, Madison Wis.) and purchased from BioCorp (Montreal). The primer sequences, and amplicon sizes are given in Table 2. The TD1 protocol for PCR amplification consisted of the following: initial denaturation for 5 min. at 94 C., followed by 17 cycles of denaturation at 95° C. for 30 sec, annealing at a temperature starting at 70° C. and ending at 54° C. (−1° C. per cycle), and 45 sec elongation at 72 C., followed by 25 cycles of denaturation at 95° C. for 30 sec., annealing at 54° C. for 30 sec. and elongation at 72° C. for 45 sec., followed by a single cycle at 72° C. for 5 min.

TABLE 2

| | | | | |
|---|---|---|---|---|
| Amplicon | Forward primer | Reverse primer | Size (bp) | Protocol |

| | | | | |
|---|---|---|---|---|
| XH18 × 01e | XGR0720: (SEQ ID NO:24) | XGR0721: (SEQ ID NO:25) | 677 | TD1 |
| | TTCCAGAAGCA\overline{TT}GTTATTTATTT | CCCCCTTGTAGTGGCTTCT | | |
| XH18 × 01b | XGR0722: (SEQ ID NO:26) | XGR0723: (SEQ ID NO:27) | 638 | TD1 |
| | CACCAGAGGCCGTAGTTATGTTG | TTGAGGAGGCAGTTCTTCTTGATT | | |
| XH18 × 01c | XGR0724: (SEQ ID NO:28) | XGR0725: (SEQ ID NO:29) | 669 | TD1 |
| | GCGCCTGCTGTGTTAACTCATAA | CCAAAGATGGGGAAACTCTACTGA | | |

Samples were amplified using Taq polymerase (Qiagen) on a PE 9700 thermocycler. All amplified fragments were tested by agarose gel electrophoresis. Samples tested were HSAN4-70 (affected homozygous), HSAN4-371 (normal control from same pedigree) and HSAN5-302 (affected). Amplified products and sequencing primers were sent to the Montreal Genome Centre sequencing facility for sequence determination. Traces were then aligned using a genomic sequence contig constructed in SeqManII (DNASTAR), and variations identified and annotated to an Excel spreadsheet. Publicly available predicted SNPs (from NCBI dbSNP) were identified, or those found in the cDNA contig were noted. Sequence variants were documented by printing the screen image in MS PhotoEditor, selecting the relevant sequence trace and pasting into MSWord to make a Figure.

Identification of mutations in HSN2: Three separate mutations were identified that were predicted to gravely affect the ORF of the HSN2 gene (see Table 3, SEQ ID NO: 3 to 5). A deletion of an A at position 594 (relative to the start ATG), found homozygous in the HSAN4-70 sample, is predicted to cause a frameshift in codon 198 leading to premature truncation of a 206 aa peptide. Two different mutations were found in the HSAN5-302 sample. The first is a 1 bp insertion of an A between bases 918-919, causing a frameshift in codon 307, and leading to premature truncation of a 318 aa peptide. The second is a C>T change at position 943 that changes codon 315 (CAG, encoding Glutamine) to a TAG stop codon, therefore prematurely truncating the protein at 314 amino acids. Each of these mutations was not found in the control sample, dbSNP database, nor available genomic and EST sequences.

TABLE 3
Sequence ID Numbers

| SEQ ID NO. | Description |
|---|---|
| 1 | Wild-Type Human HSN2 putative cDNA (ends based |
| | on human ESTs) |
| 2 | Wild-Type Human sensorin |
| 3 | Mutation 1 - Amino acid sequence of truncated |
| | human sensorin in HSAN4-70 |
| 4 | Mutation 2 - Amino acid sequence of truncated |
| | human sensorin in HSAN4-302 |
| 5 | Mutation 3 - Amino acid sequence of truncated |
| | human sensorin in HSAN4-302 |
| 6 | Genomic promoter sequence for human HSN2 |
| 7 | Wild-Type Mouse HSN2 cDNA predicted (5′ and |
| | 3′ end not determined) |
| 8 | Wild-Type Mouse sensorin amino acid sequence. |
| | 433 amino acids |
| 9 | Wild-type Rat HSN2 nucleic acid sequence. |
| | (genomic sequence defined by 5′ and 3′ ends |
| | in EST BF522762/AI578184, contains polyA tail) |
| 10 | Wild-type rat sensorin amino acid sequence. 434 |
| | amino acids |
| 11 | Wild-type Fugu sensorin amino acid sequence |
| 12 | Genomic structure and sequence of the human HSN2 |
| 13 | Partial wild-type amino acid sequence of |
| | Tetraodon sensorin |
| 14 | Human cDNA; mutation 1, c.594delA |
| 15 | Human cDNA; mutation 2, c.918insA |
| 16 | Human cDNA; mutation 3, c.943C > T |
| 17 | Pig HSN2 Nucleotide Sequence |
| 18 | Pig HSN2 Amino Acid Sequence |
| 18 | Human HSN2 translated from 2^{nd} ATG |
| 20 | Human HSN2 translated from 3^{rd} ATG |
| 21 | Mouse HSN2 translated from 2^{nd} ATG |
| 22 | Mouse HSN2 translated from 3^{rd} ATG |
| 23 | Rat HSN2 translated from 2^{nd} ATG |

SEQ ID NO: 17 is the complete sequence of pig (Sus scrofa) cDNA clone which was determined from clone MI-P-AY1-nrg-c-02-0-UI generated by Pig Genome Project and partially sequenced with Genbank accession number BI399422. SEQ ID NO: 18 is the sequence for the pig HSN polypeptide.

Analysis of SEQ ID NO: 19 to 23 shows that the 3rd ATG is closest to the optimal Kozak consensus C-C-[AG]-C-CA-T-G-G context for initiation of translation, but is not conserved in rat Hsn2 genomic sequence.

What is claimed is:

1. A method for identifying an agent that modulates the activity of the HSN2 gene, comprising:
a) contacting a test compound with a cell that expresses an HSN2 gene;
b) determining a change in the expression of said gene as a result of said contacting,
wherein said determined change in expression of the gene indicates modulation, thereby identifying the test compound as an agent that modulates the activity of an HSN2 gene.

2. The method of claim 1 wherein said HSN2 gene is a mammalian HSN2I gene.

3. The method of claim 2 wherein the mammal is mouse, rat or human.

4. The method of claim 1 wherein said modulation is a decrease in expression.

5. The method of claim 1 wherein said cell is a mammalian cell.

6. The method of claim 5 wherein said cell is a recombinant cell.

7. The method of claim 5 wherein said cell is a cell of the nervous system.

8. The method of claim 1 wherein said gene comprises a polynucleotide corresponding to a polynucleotide having a nucleotide sequence selected from the group consisting of SEQ ID NO: 1, 7 and 9.

9. A method for identifying an agent that modulates HSN2 gene, comprising:
(a) contacting a test compound with a genetic construct comprising a reporter gene operably linked to an HSN2 promoter under conditions where the reporter gene is expressed;
(b) determining a change in expression of the reporter gene as a result of said contacting,
wherein a determined change in expression indicates modulation, thereby identifying the test compound as an agent that modulates HSN2 gene.

10. The method of claim 9 wherein said modulation is a decrease in expression of said reporter gene.

11. The method of claim 9 wherein said HSN2 promoter is a mammalian HSN2 promoter.

12. The method of claim 11 wherein the mammal is mouse, rat or human.

13. The method of claim 9 wherein said promoter is the promoter of SEQ ID NO: 6.

14. The method of claim 9 wherein said genetic construct is present in a cell.

15. The method of claim 9 wherein said cell is a mammalian cell.

16. The method of claim 9 wherein said cell is a recombinant cell.

17. The method of claim 15 wherein said cell is a nervous system cell.

18. A method for identifying an agent that modulates the activity of an HSN2-encoded protein, comprising:
a) contacting a test compound with an HSN2-encoded polypeptide under conditions where said polypeptide is active; and
b) determining a change in the activity of said polypeptide as a result of said contacting;
wherein said determined change in activity indicates modulation, thereby identifying the test compound as an agent that modulates the activity of HSN2-encoded protein.

19. The method of claim 18 wherein said HSN2-encoded protein is a mammalian HSN2-encoded polypeptide.

20. The method of claim 19 wherein the mammal is mouse, rat or human.

21. The method of claim 18 wherein said modulation is a decrease in activity.

22. The method of claim 18 wherein said protein is present in a mammalian cell.

23. The method of claim 22 wherein said cell has been engineered to contain said protein.

24. The method of claim 23 wherein said cell was engineered by genetic engineering.

25. The method of claim 23 wherein said cell does not contain said protein absent said engineering.

26. The method of claim 22 wherein said cell is a cell of the nervous system.

27. The method of claim 22 wherein said protein is encoded by a polynucleotide having a sequence selected from the group consisting of SEQ ID NO: 1, 7, and 9.

28. The method of claim 22 wherein said protein comprises an amino acid sequence of SEQ ID NO: 2, 8, 10 or 11.

29. A method of treating a pain-related disorder comprising administering to an animal in need thereof a therapeutically effective amount of an HSN2 modulator.

30. The method of claim 29 wherein said HSN2 modulator exhibits modulating activity in an assay method of one of claims 1 to 28.

31. The method of claim 29 wherein said agent was first identified as an HSN2 modulator using an assay method of one of claims 1 to 28.

32. A method for treating an HSN2-related disorder comprising administering to a person in need thereof an effective amount of a selective sensorin agonist or antagonist, or a pharmaceutically acceptable salt thereof, or a pharmaceutical composition containing said agonist or antagonist.

33. The method according to claim 32 wherein said disorder is pain, neuropathy or a related disorder.

34. The method of claim 33, wherein said pain disorder is selected from the group consisting of inflammatory pain, a neuralgia, a nerve entrapment syndrome, and pain associated with a musculoskeletal disorder.

35. A method of diagnosing the presence of, or risk of developing, an HSN2-related disorder comprising determining the presence of a mutation in the nucleic acid sequence of the HSN2 gene of said individual or in the amino acid sequence of an HSN2-encoded protein of said individual.

36. A method for identifying an agent that modulates HSN2 gene activity or sensorin activity, comprising: (a) contacting a test compound with a cell expressing HSN2; and (b) determining a change in expression of a HSN2 nucleic acid or the activity of sensorin, wherein said change indicates modulation thereby identifying the test compound as an agent that modulates HSN2 gene activity or sensorin activity.

37. The method of claim 36, wherein the test compound is a small molecule.

38. The method of claim 36, wherein the test compound is an anti-sensorin antibody.

39. The method of claim 36, wherein the test compound is an antisense HSN2 nucleic acid molecule.

40. The method of claim 36, wherein the test compound is a HSN2 ribozyme.

41. An isolated polynucleotide comprising a nucleotide sequence with at least 60% identity to a sequence.selected from the group consisting of SEQ ID NO: 1, 6, 7, 9 and 12 wherein said isolated polynucleotide encodes a polypeptide that binds to an antibody specific for a polypeptide having the amino acid sequence of SEQ ID NO: 2.

42. The isolated polynucleotide of claim 41 wherein said identity is at least 70%.

43. The isolated polynucleotide of claim 41 wherein said identity is at least 78%.

44. The isolated polynucleotide of claim 41 wherein said identity is at least 90%.

45. The isolated polynucleotide of claim 41 wherein said identity is at least 95%.

46. The isolated polynucleotide of claim 41 wherein said identity is at least 98%.

47. The isolated polynucleotide of claim 41 wherein said polynucleotide has a sequence selected from the group consisting of SEQ ID NO: 1, 6, 7, 9 and 12.

48. An isolated polypeptide comprising an amino acid sequence with at least 90% identity to an amino acid sequence selected from the group consisting of SEQ ID NO: 2, 8, 10, 11 and 13 wherein said polypeptide binds to an antibody specific for a polypeptide having the amino acid sequence of SEQ ID NO: 2.

49. The isolated polypeptide of claim 48 wherein said percent identity is at least 95%.

50. The isolated polypeptide of claim 48 wherein said percent identity is at least 98%.

51. An isolated polypeptide comprising a polypeptide having an amino acid sequence selected from the group consisting of SEQ ID NO: 2, 3, 4, 5, 8, 10, 11 and 13.

52. A method for identifying an analgesic agent, comprising:
a) administering to an animal an agent found to modulate HSN2 gene or HSN2-encoded polypeptide activity, and
b) determining in said animal a decrease in response to a pain stimulus as a result of said administering,
wherein a decrease in response to said pain stimulus indicates analgesic activity, thereby identifying said agent as an analgesic agent.

53. The method of claim 52 wherein said animal is a mammal.

54. The method of claim 53 wherein said mammal is a mouse, a rat or a human being.

55. A method for producing test data with respect to the gene modulating activity of a compound comprising:
(a) contacting a test compound with a HSN2 gene under conditions where said gene is being expressed;
b) determining a change in the expression of said gene as a result of said contacting, and
(c) producing test data with respect to the gene modulating activity of said test compound based on a change in the expression of the determined gene as a result of said contacting.
